(19) United States
(12) Patent Application Publication
(10) Pub. No.: US 2026/0159504 A1
(43) Pub. Date: Jun. 11, 2026

(54) SOLID STATE FORMS OF DEUCRAVACITINIB, DEUCRAVACITINIB HCL AND PROCESS FOR PREPARATION OF DEUCRAVACITINIB AND INTERMEDIATES

(71) Applicant: ASSIA CHEMICAL INDUSTRIES LTD., TEL AVIV (IL)

(72) Inventors: Ivana Sagud, Gornja Stubica (HR); Tomislav Biljan, Krizevci (HR); Zrinka Mastelic Samardzic, Zagreb (HR); Tina Dancevic Momcilovic, Sveti Ivan Zelina (HR); Anabela Ljubic, Ivanec (HR); Marija Kos, Sveti Durd (HR); Dijana Skalec Samec, Jastrebarsko (HR); Valentina Travancic, Zagreb (HR)

(21) Appl. No.: 18/707,948

(22) PCT Filed: Dec. 1, 2022
(86) PCT No.: PCT/US2022/051466

§ 371 Date: May 7, 2024

Related U.S. Application Data
(60) Provisional application No. 63/284,703, filed on Dec. 1, 2021.
(60) Provisional application No. 63/290,744, filed on Dec. 17, 2021.
(60) Provisional application No. 63/310,211, filed on Feb. 15, 2022.
(60) Provisional application No. 63/322,687, filed on Mar. 23, 2022.
(60) Provisional application No. 63/347,063, filed on May 31, 2022.
(60) Provisional application No. 63/348,513, filed on Jun. 3, 2022.
(60) Provisional application No. 63/358,286, filed on Jul. 5, 2022.
(60) Provisional application No. 63/418,691, filed on Oct. 24, 2022.

(51) Int. Cl. C07D 403/12; C07B 59/00

(52) U.S. Cl. CPC C07D 403/12; C07B 59/002

(57) ABSTRACT

The present disclosure encompasses solid state forms of Deucravacitinib:L-tartaric acid or Deucravacitinib-L-tartrate, as well as processes for preparation of Deucravacitinib and intermediates thereof.

### FIELD OF THE DISCLOSURE

The present disclosure encompasses solid state forms of Deucravacitinib and of Deucravacitinib HCl, in embodiments the present disclosure encompasses processes for preparation thereof, and pharmaceutical compositions thereof. The present disclosure encompasses Deucravacitinib salts and solid state forms thereof, as well as processes for preparation thereof, and pharmaceutical compositions thereof. The present disclosure further relates to a process for the preparation of Deucravacitinib and salts thereof, as well as to intermediates for the preparation of Deucravacitinib and salts thereof.

### BACKGROUND OF THE DISCLOSURE

Deucravacitinib has the chemical name 6-[(Cyclopropylcarbonyl)amino]-4-[[2-methoxy-3-(1-methyl-1H-1,2,4-triazol-3-yl)phenyl]amino]-N-(methyl-d3)-3-pyridazinecarboxamide and the following chemical structure.

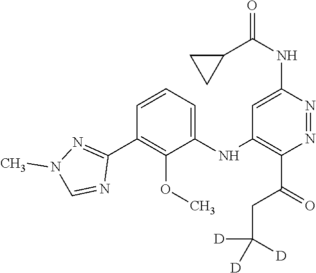 [SMILES: [2H]C([2H])([2H])CC(=O)c1nnc(NC(=O)C2CC2)cc1Nc1cccc(-c2ncn(C)n2)c1OC]

Deucravacitinib is a selective tyrosine kinase 2 (TYK2) inhibitor which is investigated and developed for the treatment of psoriasis.

Deucravacitinib is described in International Publication No. WO 2014074661. In this publication, Deucravacitinib is prepared by a process which includes reacting a triazole-aniline compound (which will be described herein below as Compound IV) with a methyl deuterium carboxamide-pyridazine compound, i.e., the methyl deuterium is introduced at an early step of the process.

J. Med. Chem. 2019, 62, 8953-8972 describes a similar process for Deucravacitinib. International Publication Nos. WO 2018183649 and WO 2018183656 describe a synthetic process for Deucravacitinib that uses a different rearrangement of steps, in which an ethyl-ester pyridazine compound is first hydrolysed to an acid or a salt compound, which is then reacted with the triazole-aniline compound. In this process, the methyl deuterium is introduced into the molecule at a later stage, by reaction of a later intermediate with deuterated methylamine.

The present disclosure further provides a simple and efficient process which avoids the use of metal counter ions and without the need of the additional step of the ester hydrolysis. The present disclosure also provides a simple and cost-efficient process for preparation of a Deucravacitinib intermediate (which will be described herein below as Compound V), which can be used in another synthetic process, such as the process described in WO 2014074661.

International Publication Nos. WO 2018183656 and WO 2021143498 describe polymorphs of Deucravacitinib.

International Publication Nos WO 2019232138 and WO 2021143430 describe Deucravacitinib HCl salt and polymorphs thereof. International Publication No. WO 2020251911 describe polymorphs of Deucravacitinib mesylate and Deucravacitinib sulfonate.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Deucravacitinib and of Deucravacitinib HCl, as well as salts of Deucravacitinib and their solid state forms.

### SUMMARY OF THE DISCLOSURE

The present disclosure encompasses solid state forms of Deucravacitinib and of Deucravacitinib HCl, in embodiments the present disclosure encompasses processes for preparation thereof, and pharmaceutical compositions thereof. The present disclosure encompasses Deucravacitinib salts and solid state forms thereof, as well as processes for preparation thereof, and pharmaceutical compositions thereof.

These solid state forms of the present disclosure, particularly crystalline polymorphs, of Deucravacitinib and of Deucravacitinib HCl, and crystalline polymorphs of Deucravacitinib salts can be used to prepare other solid state forms of Deucravacitinib, Deucravacitinib salts or co-crystals and their solid state forms.

The present disclosure also provides uses of the said solid state form of Deucravacitinib and of Deucravacitinib HCl and of Deucravacitinib salts in the preparation of other solid state forms of Deucravacitinib, Deucravacitinib of Deucravacitinib salts or co-crystals salts thereof.

The present disclosure provides solid state forms, particularly crystalline polymorphs, of Deucravacitinib and of Deucravacitinib HCl and of Deucravacitinib salts for use in medicine, including for the treatment of patients with psoriasis.

The present disclosure also encompasses the use of the solid state forms, particularly crystalline polymorphs, of Deucravacitinib and of Deucravacitinib HCl and crystalline polymorphs of Deucravacitinib salts of the present disclosure for the preparation of pharmaceutical compositions and/or formulations. In embodiments, solid state forms, particularly crystalline polymorphs, of Deucravacitinib and of Deucravacitinib HCl of the present disclosure are used to prepare oral dosage forms of Deucravacitinib and of Deucravacitinib HCl.

In another aspect, the present disclosure provides pharmaceutical compositions, such as oral dosage forms of Deucravacitinib and of Deucravacitinib HCl, comprising the solid state forms, particularly crystalline polymorphs, of Deucravacitinib and/or of Deucravacitinib HCl or of Deucravacitinib salts according to the present disclosure.

The present disclosure includes processes for preparing the above mentioned pharmaceutical compositions. The processes include combining any one or a combination of the solid state forms, particularly crystalline polymorphs, of Deucravacitinib and of Deucravacitinib HCl or crystalline polymorphs of Deucravacitinib salts with at least one pharmaceutically acceptable excipient.

The solid state forms, particularly crystalline polymorphs, of Deucravacitinib and of Deucravacitinib HCl and crystalline polymorphs of Deucravacitinib salts as defined herein and the pharmaceutical compositions or formulations of solid state forms, particularly crystalline polymorphs, of Deucravacitinib and of Deucravacitinib HCl may be used as medicaments, such as for the treatment of psoriasis.

The present disclosure also provides methods of treating psoriasis, by administering a therapeutically effective amount of any one or a combination of the solid state forms, particularly crystalline polymorphs, of Deucravacitinib and of Deucravacitinib HCl or crystalline polymorphs of Deucravacitinib salts of the present disclosure, or at least one of the above pharmaceutical compositions, to a subject suffering from psoriasis. The present disclosure also provides uses of solid state forms, particularly crystalline polymorphs, of Deucravacitinib, Deucravacitinib HCl and crystalline polymorphs of Deucravacitinib salts of the present disclosure, or at least one of the above pharmaceutical compositions, for the manufacture of medicaments for treating patients with psoriasis.

The present disclosure further relates to a process for the preparation of Deucravacitinib and salts thereof, as well as to intermediates for the preparation of Deucravacitinib and salts thereof. The processes disclosed herein involve converting compound XIII, XIV or XVII as described below to Deucravacitinib. In aspects, the process utilizes a reaction of Compound XI and IV to obtain Compound XIII, which can be converted to Deucravacitinib. The Intermediates and process are described herein below.

### BRIEF DESCRIPTION OF THE DRAWINGS

FIG. 1 shows a characteristic X-ray powder diffraction pattern (XRPD) of crystalline Deucravacitinib form B1.

FIG. 2 shows a characteristic XRPD of crystalline Deucravacitinib Form B2.

FIG. 3 shows a characteristic XRPD of crystalline Deucravacitinib HCl Form H1.

FIG. 4 shows a characteristic XRPD of crystalline Deucravacitinib HCl Form H2.

FIG. 5 shows a characteristic XRPD of crystalline Deucravacitinib HCl Form H4.

FIG. 6 shows a characteristic XRPD of crystalline Deucravacitinib HCl Form H8.

FIG. 7 shows a characteristic XRPD of crystalline Deucravacitinib HCl:L-Malic acid Form H6.

FIG. 8 shows a characteristic XRPD of crystalline Deucravacitinib HCl:urea Form H5.

FIG. 9 shows a characteristic XRPD of crystalline Deucravacitinib HCl:urea Form H7.

FIG. 10 shows a characteristic XRPD of crystalline Deucravacitinib HCl:L-Malic acid Form H9.

FIG. 11 shows a characteristic XRPD of crystalline Deucravacitinib HBr Form T1.

FIG. 12 shows a characteristic XRPD of crystalline Deucravacitinib HBr Form T2.

FIG. 13 shows a characteristic XRPD of crystalline Deucravacitinib EDSA Form T5.

FIG. 14 shows a characteristic XRPD of crystalline Deucravacitinib ESA Form T11.

FIG. 15 shows a characteristic XRPD of crystalline Deucravacitinib HCl:urea Form H5, prepared according to example 18.

FIG. 16 shows a characteristic XRPD of crystalline Deucravacitinib HCl Form H1, prepared according to example 19.

FIG. 17 shows a characteristic XRPD of crystalline Deucravacitinib Form A, according to WO 2018183656.

FIG. 18 shows a characteristic XRPD of crystalline Deucravacitinib:L-Malic acid Form Im.

FIG. 19 shows a characteristic XRPD of crystalline Deucravacitinib:L-tartaric acid Form It.

FIG. 20 shows a characteristic XRPD of crystalline Deucravacitinib:maleic acid Form M1.

FIG. 21 shows a characteristic XRPD of crystalline Deucravacitinib:L-Malic acid Form IIm.

FIG. 22 shows a characteristic XRPD of crystalline Deucravacitinib:L-Malic acid Form IIIm.

FIG. 23 shows a characteristic XRPD of crystalline Deucravacitinib:L-tartaric acid Form IIt.

FIG. 24 shows a characteristic X-ray powder diffraction pattern (XRPD) of crystalline Compound XIII Form A.

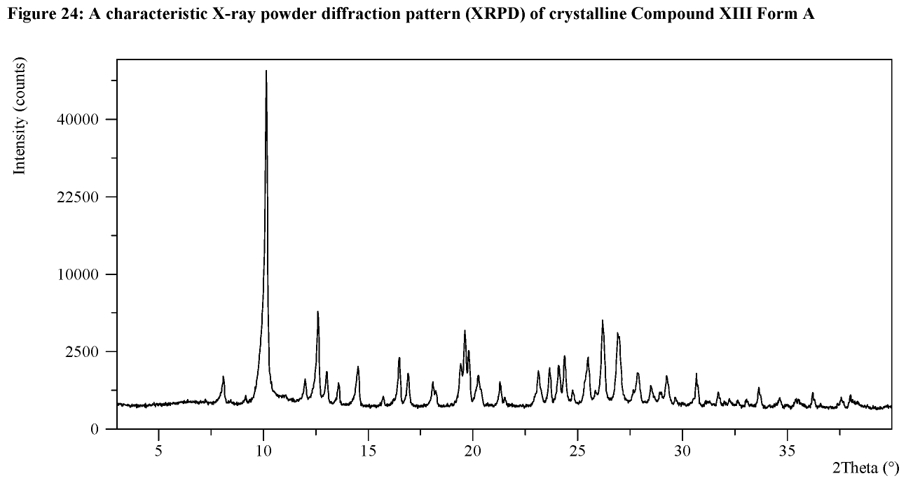

FIG. 25 shows a characteristic XRPD of crystalline Compound XIV Form 1.

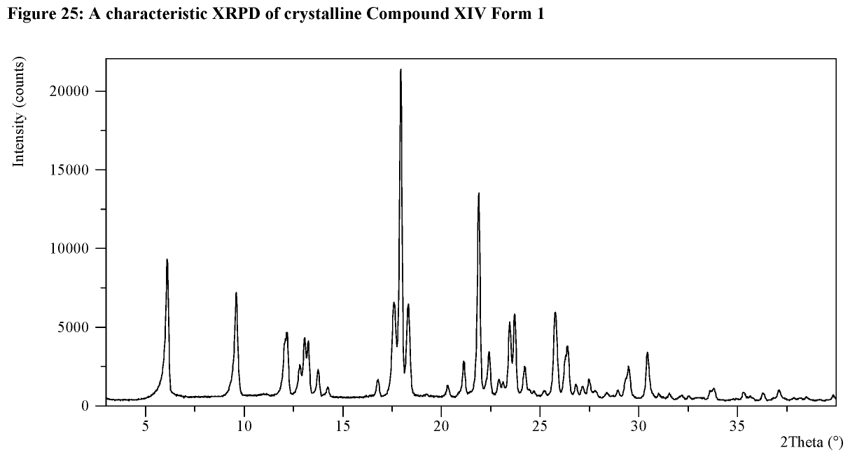

FIG. 26 shows a characteristic XRPD of crystalline Compound XVII Form B.

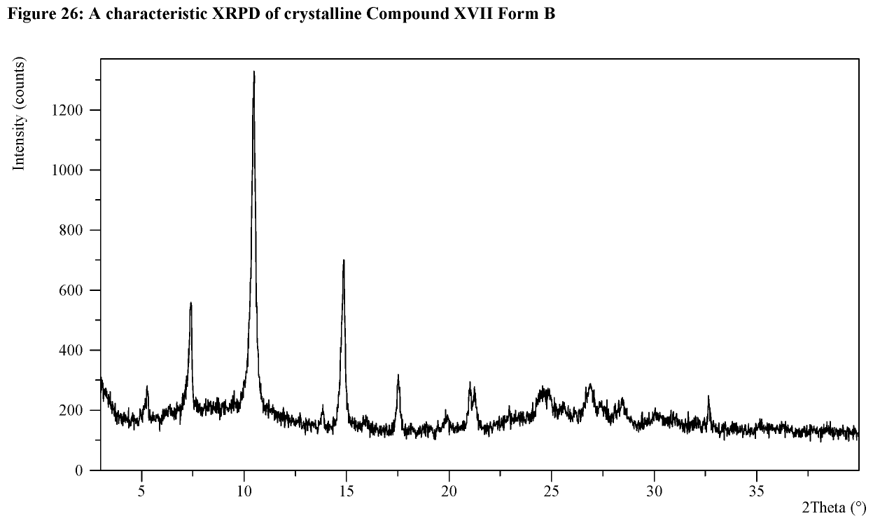

FIG. 27 shows a characteristic solid-state ¹³C NMR spectrum of Deucravacitinib and L-tartaric acid co-crystal form It.

FIG. 28 shows a characteristic solid-state ¹⁵N NMR spectrum of Deucravacitinib and L-tartaric acid co-crystal form It.

### DETAILED DESCRIPTION OF THE DISCLOSURE

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Deucravacitinib referred to herein as being characterized by graphical data “as depicted in” or “as substantially depicted in” a Figure will thus be understood to include any crystal forms of Deucravacitinib characterized with the graphical data having such small variations, as are well known to the skilled person, in comparison with the Figure.

As used herein, and unless stated otherwise, the term “anhydrous” in relation to crystalline forms of Deucravacitinib or Deucravacitinib HCl, relates to a crystalline form of Deucravacitinib which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Deucravacitinib or Deucravacitinib HCl of the present disclosure corresponds to a crystalline polymorph of Deucravacitinib or Deucravacitinib HCl that is physically separated from the reaction mixture in which it is formed.

As used herein, unless stated otherwise, the XRPD measurements are taken using copper Kα1 radiation wavelength 1.5418 Å. XRPD peaks reported herein are measured using CuK α1 radiation, λ=1.5418 Å, typically at a temperature of 25±3° C.

As used herein, unless stated otherwise, ¹H, ¹³C and ¹⁵N NMR reported herein are measured at 400 MHz, 100 MHz and 40 MHz, preferably at a temperature of at 293 K±3° C.

A thing, e.g., a reaction mixture, may be characterized herein as being at, or allowed to come to “room temperature” or “ambient temperature,” often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added (e.g. adding methyl tert-butyl ether (MTBE) (1.5 v/v) to a 100 ml reaction mixture would indicate that 150 mL of MTBE was added).

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

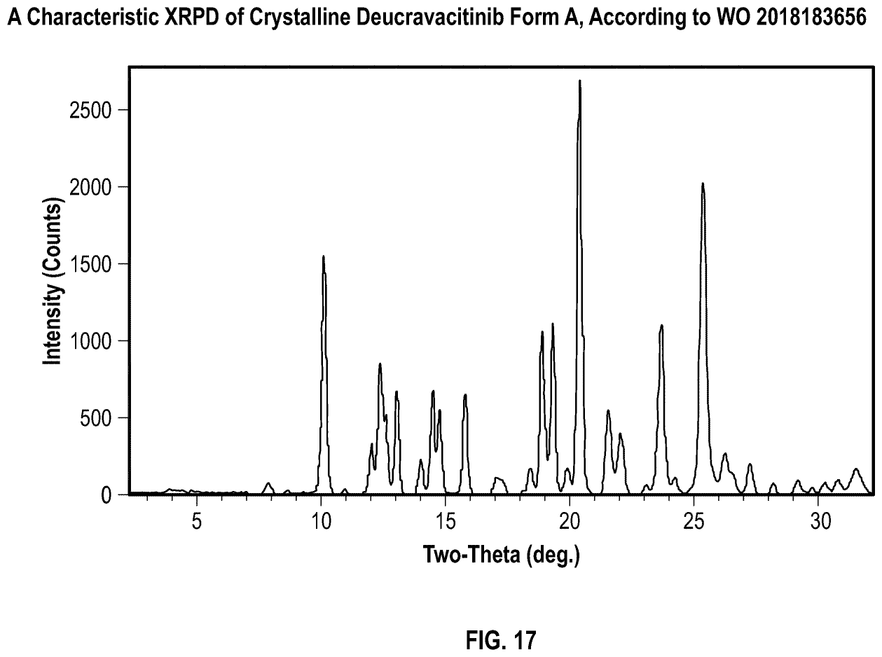

As used herein, the term crystalline Deucravacitinib form A or form A of Deucravacitinib refers to crystalline form of Deucravacitinib as described in International Publication WO 2018183656. Crystalline Deucravacitinib form A has a characteristic XRPD as shown in FIG. 17.

As used herein, crystalline Deucravacitinib HCl:L-Malic acid is a distinct molecular species. Crystalline Deucravacitinib HCl:L-Malic acid may be a co-crystal of Deucravacitinib HCl and L-Malic acid. Alternatively crystalline Deucravacitinib HCl:L-Malic acid may be a salt, i.e. Deucravacitinib HCl:L-Malate.

As used herein, crystalline Deucravacitinib HCl:Urea is a distinct molecular species. Crystalline Deucravacitinib HCl:Urea may be a co-crystal of Deucravacitinib HCl and Urea.

As used herein, crystalline Deucravacitinib:L-Malic acid, i.e. Deucravacitinib free base and L-Malic complex, is a distinct molecular species. Crystalline Deucravacitinib:L-Malic acid may be a co-crystal of Deucravacitinib and L-Malic acid. Alternatively, crystalline Deucravacitinib:L-Malic acid may be a salt, i.e., Deucravacitinib L-Malate.

As used herein, crystalline Deucravacitinib:L-tartaric acid, i.e. Deucravacitinib free base:L-tartaric acid is a distinct molecular species. Crystalline Deucravacitinib:L-tartaric acid may be a co-crystal of Deucravacitinib and L-tartaric acid. Alternatively crystalline Deucravacitinib:L-tartaric acid may be a salt, i.e. Deucravacitinib:L-tartrate. Preferably, Deucravacitinib:L-tartaric acid is a complex.

As used herein, crystalline Deucravacitinib:maleic acid, i.e. Deucravacitinib free base:maleic acid is a distinct molecular species. Crystalline Deucravacitinib:maleic acid may be a co-crystal of Deucravacitinib and maleic acid. Alternatively crystalline Deucravacitinib:L-maleic acid may be a salt, i.e. Deucravacitinib maleate.

### Processes for the Preparation of Deucravacitinib and Salts Thereof

The present disclosure relates to a process for the preparation of Deucravacitinib and salts thereof, as well as to intermediates for the preparation of Deucravacitinib and salts thereof.

The present disclosure comprises a process for preparing Deucravacitinib or salt thereof comprising:

### (A) Reacting:

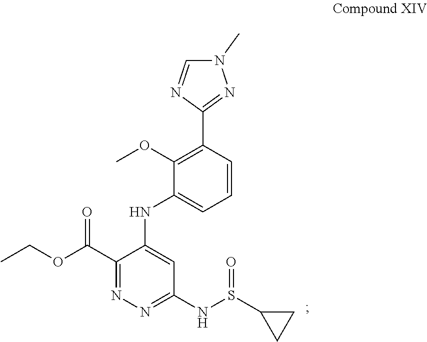 [SMILES: CCOC(=O)c1nnc(NS(=O)C2CC2)cc1Nc1cccc(-c2ncn(C)n2)c1OC]

### (ii) Compound XIII:

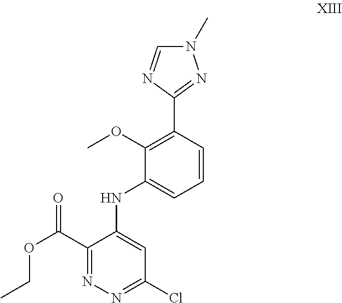 [SMILES: CCOC(=O)c1nnc(Cl)cc1Nc1cccc(-c2ncn(C)n2)c1OC]

with deuterated methylamine or a salt thereof, preferably deuterated methylamine hydrochloride; wherein in the case of (ii), the resulting Compound XVII formed by the reaction of Compound XIII with the deuterated methylamine or salt thereof:

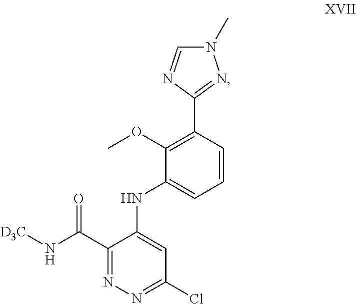 [SMILES: [2H]C([2H])([2H])NC(=O)c1nnc(Cl)cc1Nc1cccc(-c2ncn(C)n2)c1OC]

is reacted with cyclopropanecarboxamide;
or

### (B) Reacting Compound XVII:

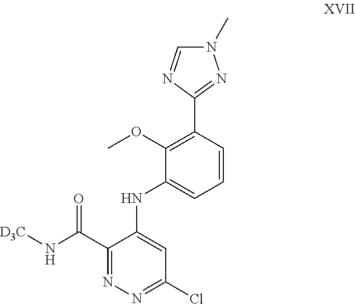 [SMILES: [2H]C([2H])([2H])NC(=O)c1nnc(Cl)cc1Nc1cccc(-c2ncn(C)n2)c1OC]

with cyclopropanecarboxamide in the presence of {(R)-1-[(Sp)-2-(dicyclohexyl phosphino)ferrocenyl]ethyldi-tert-butylphosphine}[2-(2-amino-1,1-biphenyl)]palladium(II) methanesulfonate, Pd(OAc)₂, and an alkali metal base, preferably potassium carbonate.

In option (A)(i), wherein Compound XIV is reacted with deuterated methylamine or a salt thereof, preferably deuterated methylamine hydrochloride to form Compound XVII which is reacted with cyclopropanecarboxamide to form Deucravacitinib, the compound XIV may be prepared by reacting Compound XIII:

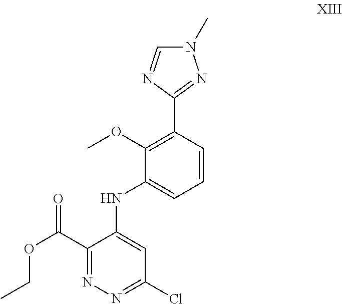 [SMILES: CCOC(=O)c1nnc(Cl)cc1Nc1cccc(-c2ncn(C)n2)c1OC]

with cyclopropanecarboxamide. The resulting Deucravacitinib may be reacted with an acid to form an acid addition salt thereof.

In option (A)(ii), Compound XIII is reacted with deuterated methylamine or a salt thereof, preferably deuterated methylamine hydrochloride to form Compound XVII:

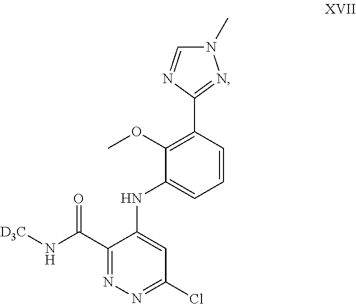 [SMILES: [2H]C([2H])([2H])NC(=O)c1nnc(Cl)cc1Nc1cccc(-c2ncn(C)n2)c1OC]

and reacting Compound XVII with cyclopropanecarboxamide. The resulting Deucravacitinib may be reacted with an acid to form an acid addition salt thereof.

In Option (B), wherein Compound XVII is reacted with cyclopropanecarboxamide in the presence of {(R)-1-[(Sp)-2-(Dicyclohexyl phosphino)ferrocenyl]ethyldi-tert-butylphosphine}[2-(2-amino-1,1-biphenyl)]palladium(II) methanesulfonate, Pd(OAc)₂, and an alkali metal base, preferably potassium carbonate, the compound XVII may be prepared by reacting Compound XIII.

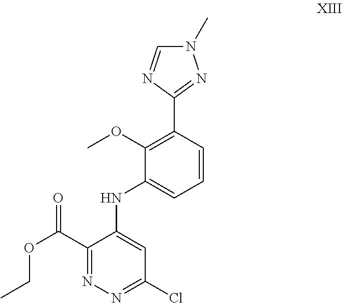 [SMILES: CCOC(=O)c1nnc(Cl)cc1Nc1cccc(-c2ncn(C)n2)c1OC]

with deuterated methylamine or a salt thereof, preferably deuterated methylamine hydrochloride. The resulting Deucravacitinib may be reacted with an acid to form an acid addition salt thereof.

In any of options (A)(i), (A)(ii) and (B), the reaction of Compound XIII or Compound XIV with deuterated methylamine or a salt thereof, preferably deuterated methylamine hydrochloride, may be carried out in a solvent and in the presence of a base. Particularly suitable solvents are polar aprotic solvents, preferably wherein the solvent is water-miscible. More preferably the solvent is selected from dimethylformamide or tetrahydrofuran. Particularly suitable bases are non-nucleophilic bases, preferably lithium bis(trimethylsilyl)amide or 2,2,6,6-tetramethylpiperidine, and more preferably wherein the base is lithium bis(trimethylsilyl)amide.

In any of options (A)(i), (A)(ii) and (B), the reaction of Compound XII or Compound XVII with cyclopropanecarboxamide may be carried out in one or more organic solvents, and in the presence of a base, and a catalyst comprising a chiral phosphine ligand and palladium salt. The base is preferably an inorganic base, optionally an alkaline metal or alkaline earth metal carbonate, preferably sodium or potassium carbonate, more particularly potassium carbonate. Preferably the chiral phosphine ligand is selected from the group consisting of: (9,9-Dimethyl-9H-xanthene-4,5-diyl)bis(diphenylphosphane) and{(R)-1-[(Sp)-2-(Dicyclohexyl phosphino)ferrocenyl]ethyldi-tert-butylphosphine}[2-(2-amino-1,1-biphenyl)]palladium(II) methanesulfonate, and preferably {(R)-1-[(Sp)-2-(Dicyclohexyl phosphino)ferrocenyl]ethyldi-tert-butylphosphine}[2-(2-amino-1,1-biphenyl)]palladium(II) methanesulfonate. The palladium salt is preferably a palladium(II) salt, and more preferably palladium acetate [Pd(OAc)₂]. Preferably, the one or more organic solvents are selected from aprotic solvents, and are more preferably selected from acetonitrile and/or toluene, and more preferably wherein the solvent is a mixture of acetonitrile and toluene.

In any embodiment of the processes disclosed herein, Compound XIII may be prepared by:
reacting Compound XI:

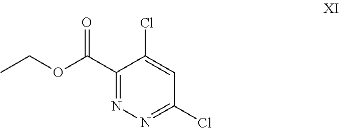

with Compound IV:

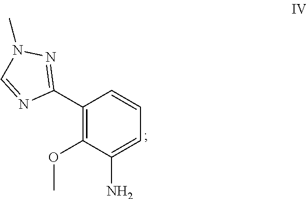

by borylation of 3-bromo-2-methoxyaniline:

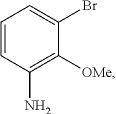

and reacting the obtained compound, with a triazole compound, preferably a 1-methyl-1H-1,2,4-triazole, having a leaving group at the 3 position, more preferably 3-bromo-1-methyl-1H-1,2,4-triazole. For example, the borylation can be carried out using bis(pinacolato)diboron or bis(neopentyl glycolato)diboron, i.e.:

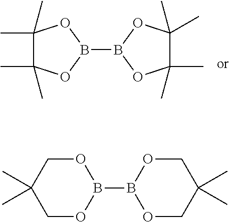

and reacting the obtained compound, i.e.:

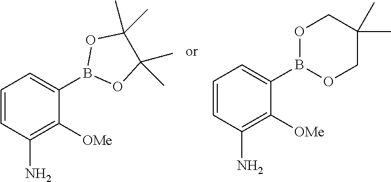 [SMILES: COc1c(N)cccc1B1OC(C)(C)C(C)(C)O1 ; COc1c(N)cccc1B1OCC(C)(C)CO1]

with a triazole compound, preferably a 1-methyl-1H-1,2,4-triazole, having a leaving group at the 3 position, more preferably 3-bromo-1-methyl-1H-1,2,4-triazole:

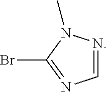

Preferably, in any embodiment of the processes disclosed herein, Compound XIII may be prepared by reacting Compound XI with Compound IV in the presence of a base, preferably a non-nucleophilic base, preferably wherein the base is lithium bis(trimethylsilyl)amide or 2,2,6,6-tetramethylpiperidine, and more preferably wherein the base is 2,2,6,6-tetramethylpiperidine. Preferably the reaction of Compound XI with Compound IV is carried out in the presence of a solvent, preferably an aprotic solvent. More preferably the solvent is selected from acetonitrile, toluene, and N,N-dimethylformamide (DMF), and particularly toluene.

In another aspect, the disclosure relates to processes for the preparation of Deucravacitinib or salt therefore, starting from a compound of formula XIV, XIII, or XVII. The process may comprise one of the following reaction sequences: XIII→XIV→I, or XIV→I, XIII→II→XIV→I, XIII→XVII→I as depicted in Scheme A:

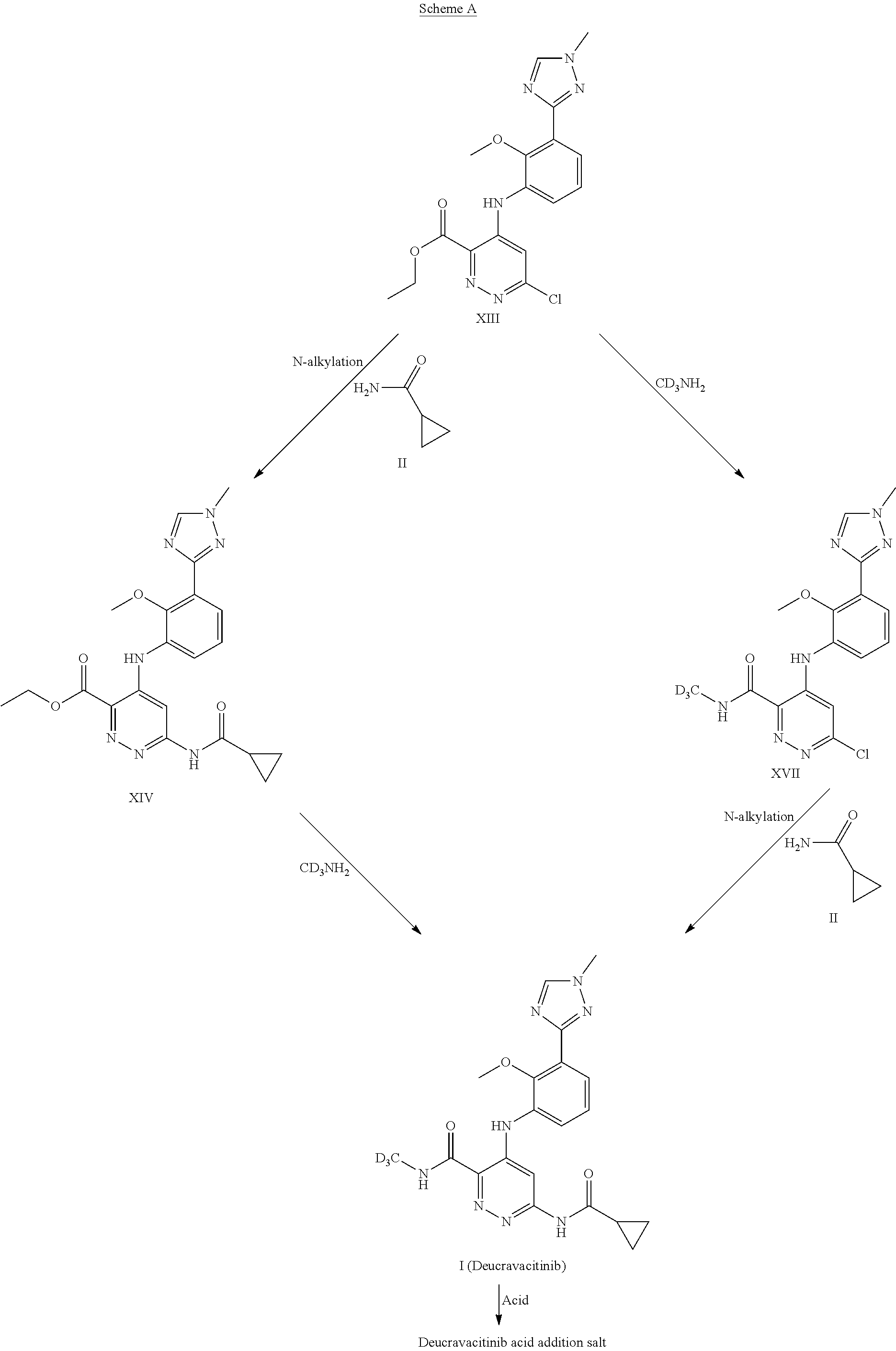

According to any embodiment of the process as described herein, the Deucravacitinib into an acid addition salt.

According to any aspect or embodiment of the processes disclosed herein, the Compound XIII may be prepared by aromatic nucleophilic substitution of Compound XI with Compound IV:

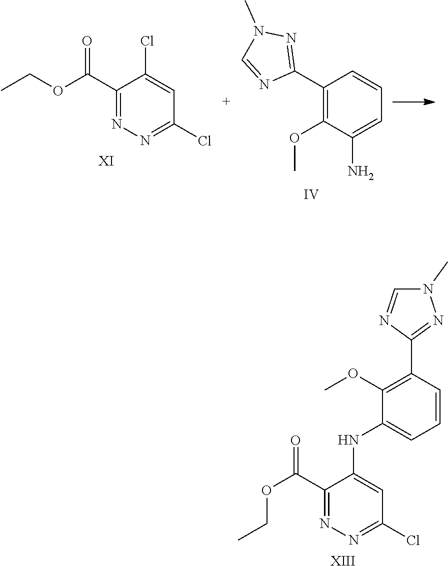

In one aspect, the present disclosure provides a process for preparing Deucravacitinib or a salt, wherein the process comprises N-alkylation of compound XIII with compound II to form the compound XIV, and reacting the compound XIV with CD₃NH₂ (i.e., aminolysis) to form Deucravacitinib.

Alternatively, the process may comprise reversing the N-alkylation and aminolysis steps, i.e., reacting the compound XIII with CD₃NH₂ to form the compound XVII, and reacting the compound XVII with compound II to form Deucravacitinib.

The present disclosure further comprises a compound selected from the group consisting of Compound XIII, Compound XIV or Compound XVII:

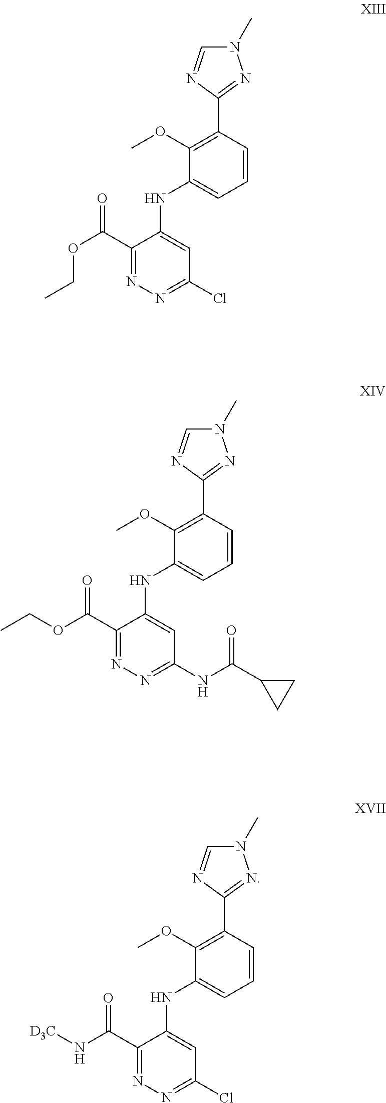

and processes for their preparation, as well as crystalline forms of Compounds XII, XIV and XVII.

Aspects of the processes of the present disclosure advantageously provide a convenient synthesis of Deucravacitinib. In aspects, the disclosure provides a process which reacts an ethyl-ester pyridazine compound, which is referred to as Compound XI:

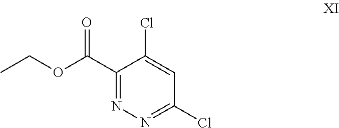

with a triazole-aniline compound, referred to as Compound IV:

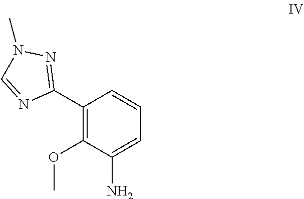

to obtain Compound XIII:

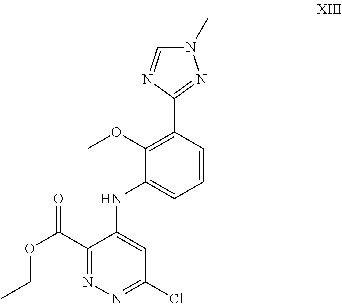 [SMILES: CCOC(=O)c1nnc(Cl)cc1Nc1cccc(-c2ncn(C)n2)c1OC]

The compound XIII may be converted to Deucravacitinib, for example as described herein.

In this process, Compound XI directly reacts with Compound IV, without the need to first hydrolyze Compound XI to a corresponding acid or metal salt.

To facilitate the reaction, a base such as Lithium bis(trimethylsilyl)amide (LiHMDS) or 2,2,6,6-tetramethylpiperidine (TMP), can be added; and the reaction may be typically carried out in the presence of one or more solvents, preferably an aprotic solvent, such as tetrahydrofuran (“THF”), acetonitrile, toluene and/or N,N-dimethylformamide (DMF), or more preferably wherein the solvent is toluene. Optionally, the reaction may be carried out using LiHMDS as base and THE as solvent. Alternatively, the reaction may be preferably carried out using TMP as base and toluene as solvent. In any embodiment, the base may be combined with the Compound IV prior to reacting with Compound XIII. In any embodiment, the reaction may be conducted at elevated temperature, for example at: about 40° C. to about 140° C., about 60° C. to about 130° C., about 80° C. to about 120° C., about 100° C. to about 115° C., or about 110° C. The reaction mixture may be cooled and the compound XIII isolated from the reaction mixture. The isolation can be advantageously achieved by adding one or more solvents, such as water and/or toluene. The solid may be isolated by any suitable method, such as decantation, filtration or centrifuge, preferably by filtration. The solid may be dried, for example under reduced pressure and/or elevated temperature (e.g. about 30° C. to about 90° C., about 35° C. to about 80° C., about 40° C. to about 70° C. or about 45° C. to about 55° C. or about 50° C. Advantageously, the compound XIII may be obtained as a solid, preferably a crystalline solid, in high yield.

The above process can further comprise reacting Compound XIII with cyclopropane carboxamide of formula II:

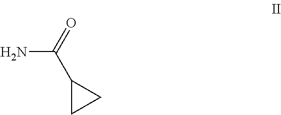

to obtain Compound XIV:

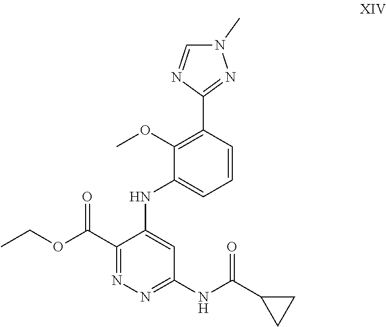 [SMILES: CCOC(=O)c1nnc(NC(=O)C2CC2)cc1Nc1cccc(-c2ncn(C)n2)c1OC]

and then reacting Compound XIV with deuterated methylamine, (CD₃NH₂), or a salt thereof, which may be referred to herein as Compound VI, to obtain Deucravacitinib (also referred to herein as Compound I).

The Deucravacitinib may be later converted to Deucravacitinib salt.

In this process, the compound XIII may be reacted with Compound II in the presence of a base. Suitably, the base may be an inorganic base, particularly an alkaline metal or alkaline earth metal carbonate, preferably sodium or potassium carbonate, more particularly potassium carbonate. The reaction may be carried out in the presence of one or more solvents, preferably aprotic solvents, particularly acetonitrile and/or toluene, and especially a mixture of acetonitrile and toluene. The reaction is preferably carried out in the presence of a chiral phosphine ligand and a palladium salt as catalysts. Suitable catalysts include (9,9-Dimethyl-9H-xanthene-4,5-diyl)bis(diphenylphosphane) (commercial name Xantphos) or {(R)-1-[(Sp)-2-(Dicyclohexyl phosphino)ferrocenyl]ethyldi-tert-butylphosphine}[2-(2-amino-1,1-biphenyl)]palladium(II) methanesulfonate. {(R)-1-[(Sp)-2-(Dicyclohexyl phosphino)ferrocenyl]ethyldi-tert-butylphosphine}[2-(2-amino-1,1-biphenyl)]palladium(II) methanesulfonate (commercial name: Josiphos SL-J009-1-G3-palladacycle) is a particularly suitable chiral phosphine ligand. Palladium acetate is a particularly preferred catalyst. Thus, in any embodiment the compound XIII can be reacted with Compound II in the presence of potassium carbonate, {(R)-1-[(Sp)-2-(Dicyclohexyl phosphino)ferrocenyl]ethyldi-tert-butylphosphine}[2-(2-amino-1,1-biphenyl)]palladium(II) methanesulfonate (commercial name: Josiphos SL-J009-1-G3-palladacycle), and palladium acetate, in acetonitrile and toluene. The reaction may be carried out at elevated temperature for example at: about 40° C. to about 100° C., about 50° C. to about 90° C., about 60° C. to about 80° C., or about 75° C. The reaction mixture may be cooled, and quenched, for example by the addition of water and aqueous ammonium chloride. The resulting compound XIV may be obtained from the quenched reaction mixture by the addition of an antisolvent, for example toluene. The isolation of compound XIV may be by any suitable method, such as decantation, filtration or centrifuge, preferably by filtration. The solid may be dried, for example under reduced pressure and/or elevated temperature (e.g. about 40° C. to about 100° C., about 50° C. to about 95° C., about 60° C. to about 90° C. or about 70° C. to about 90° C. or about 80° C. Advantageously, the compound XIV may be obtained as a solid, preferably a crystalline solid, in high yield.

As disclosed above, the compound XIV may be reacted with deuterated methylamine (Compound VI) or a salt thereof, directly via aminolysis of the ester group. Thus, Compound XIV may be reacted with deuterated methylamine (i.e. trideuteromethylamine) optionally in the form of a salt, preferably trideuteromethylamine hydrochloride. The reaction may be conducted in the presence of a nucleophilic base, preferably a strong nucleophilic base, such as lithium bis(trimethylsilyl)amide. The reaction is preferably carried out in the presence of one or more polar aprotic solvents, particularly tetrahydrofuran and/or dimethylformamide. The reaction may be advantageously carried out at ambient temperature. Preferably, the base is added to a mixture containing Compound XIII and trideuteromethylamine hydrochloride. The base and the mixture containing Compound XIII and trideuteromethylamine hydrochloride are preferably combined as solution/suspension in the polar aprotic solvent (preferably THE and/or DMF). The reaction may be carried out at a temperature of: about 10° C. to about 25° C., about 15° C. to about 25° C., or about 20° C. The reaction mixture may be quenched, for example by the addition of water and a mineral acid, particularly hydrochloric acid. The reaction mixture may be cooled for example to: about −5° C. to about 10° C., about −2° C. to about 5° C., or about 0° C. The Deucravacitinib may be isolated from the reaction mixture filtration, decantation or centrifuge, preferably by filtration. The Deucravacitinib may be dried, for example under reduced pressure and/or elevated temperature (e.g. about 40° C. to about 100° C., about 50° C. to about 95° C., about 60° C. to about 90° C. or about 70° C. to about 90° C. or about 80° C. Advantageously, Deucravacitinib may be obtained as a solid, preferably a crystalline solid, in high yield.

In another aspect, the above-described process may be generally represented by the below Scheme 1:

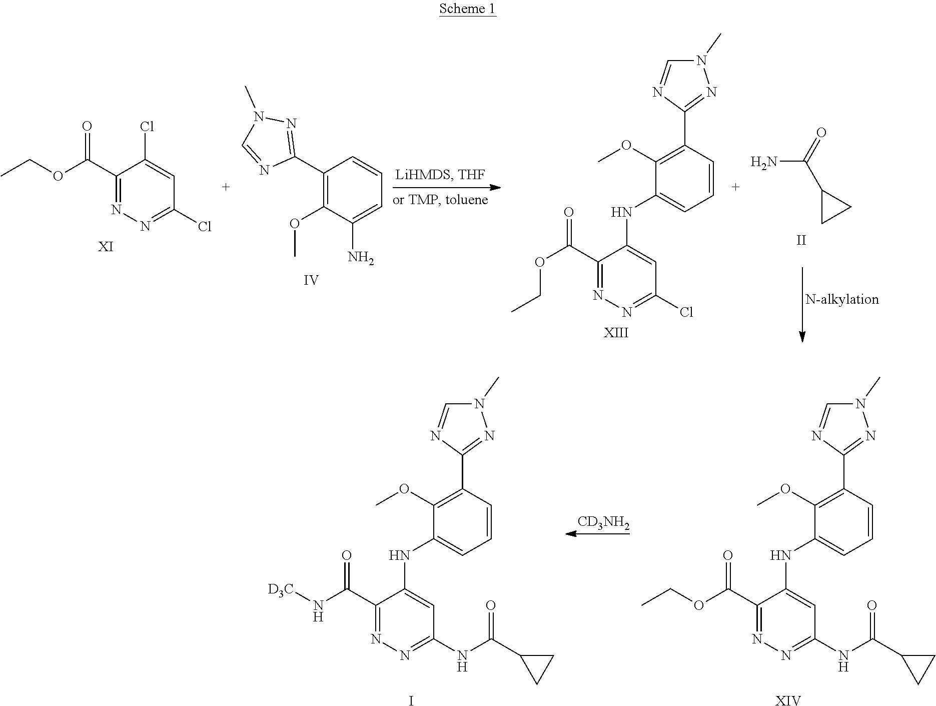

In the above Scheme 1, the reaction of Compound XI with Compound IV may be carried out using lithium bis(trimethylsilyl)amide (LiHMDS) preferably in THE (tetrahydrofuran) solvent, or using 2,2,6,6-tetramethylpiperidine (TMP) preferably in toluene solvent.

As shown in Scheme 1, the aromatic nucleophilic substitution of Compound XI with Compound IV results in the production of Compound XIII. Compound XIII is then reacted with Compound II to form Compound XIV and direct aminolysis of the ester in Compound XIV forms Deucravacitinib. The Deucravacitinib may be converted to a salt by reaction with an appropriate acid. Advantageously, the process of Scheme 1 enables the production of the intermediates as crystalline solids in high yield and high purity.

In an alternative process, Compound XIII can be converted to Deucravacitinib by a process comprising reacting compound XIII with CD₃NH₂, to obtain Compound XVII of the following structure:

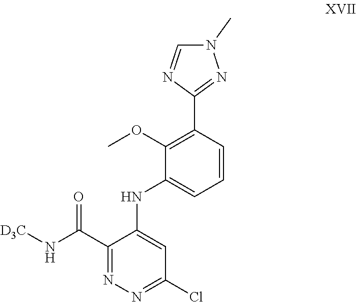 [SMILES: [2H]C([2H])([2H])NC(=O)c1nnc(Cl)cc1Nc1cccc(-c2ncn(C)n2)c1OC]

followed by reacting the obtained Compound XVII with cyclopropane carboxamide (Compound II), to obtain Deucravacitinib. Thus, in this process the aminolysis and the N-alkylation steps are reversed, i.e. the aminolysis is carried out on Compound XIII, to obtain compound XVII, and the compound XVII is reacted with cyclopropane carboxamide to obtain Deucravacitinib.

Accordingly, in the above process, the Compound XIII (prepared for example by reaction of Compound XI with Compound IV as described above) may be reacted with the cyclopropane carboxamide of formula II:

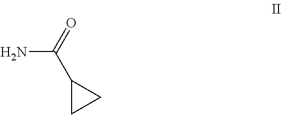

to obtain Compound XVII:

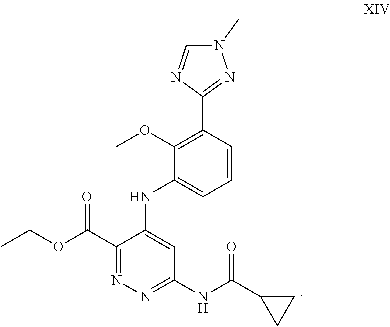 [SMILES: CCOC(=O)c1nnc(NC(=O)C2CC2)cc1Nc1cccc(-c2ncn(C)n2)c1OC]

and then reacting Compound XIV with deuterated methylamine, (CD₃NH₂), or a salt thereof, which may be referred to herein as Compound VI, to obtain Deucravacitinib (Compound I).

The Deucravacitinib may be later converted to Deucravacitinib salt.

As disclosed above, the compound XIII may be reacted with deuterated methylamine (Compound VI) or a salt thereof, directly via aminolysis of the ester group of Compound XIII. Thus, Compound XIII may be reacted with deuterated methylamine (i.e. trideuteromethylamine) optionally in the form of a salt, preferably trideuteromethylamine hydrochloride. The reaction may be conducted in the presence of a nucleophilic base, preferably a strong nucleophilic base, such as lithium bis(trimethylsilyl)amide. The reaction is preferably carried out in the presence of one or more polar aprotic solvents, particularly tetrahydrofuran. The reaction may be advantageously carried out at ambient temperature. Preferably, the base is added to a mixture containing Compound XIII and trideuteromethylamine hydrochloride. The base and the mixture containing Compound XIII and trideuteromethylamine hydrochloride are preferably combined as solution/suspension in the polar aprotic solvent (preferably THF). The reaction may be carried out at a temperature of: about 10° C. to about 25° C., about 15° C. to about 25° C., or about 20° C. The reaction mixture may be quenched, for example by the addition of water and/or aqueous ammonium chloride solution. The product Compound XVII may advantageously be isolated by extraction, and the product may be isolated as a solid by evaporation. Advantageously Compound XVII may be readily obtained as a solid, preferably a crystalline solid, in high yield.

The compound XVII may be reacted with cyclopropanecarboxamide Compound II to form Deucravacitinib. The reaction may be carried out in the presence of a base. Suitably, the base may be an inorganic base, particularly an alkaline metal or alkaline earth metal carbonate, preferably sodium or potassium carbonate, more particularly potassium carbonate. The reaction may be carried out in the presence of one or more solvents, preferably aprotic solvents, particularly acetonitrile and/or toluene, and especially a mixture of acetonitrile and toluene. The reaction is preferably carried out in the presence of a chiral phosphine ligand and a palladium salt as catalysts. Suitable catalysts include (9,9-Dimethyl-9H-xanthene-4,5-diyl)bis(diphenylphosphane) (commercial name Xantphos, 0.017 grams, 0.029 mmol) and added {(R)-1-[(Sp)-2-(Dicyclohexyl phosphino)ferrocenyl]ethyldi-tert-butylphosphine}[2-(2-amino-1,1-biphenyl)]palladium(II) methanesulfonate. {(R)-1-[(Sp)-2-(Dicyclohexyl phosphino)ferrocenyl]ethyldi-tert-butylphosphine}[2-(2-amino-1,1-biphenyl)]palladium(II) methanesulfonate (commercial name: Josiphos SL-J009-1-G3-palladacycle) is a particularly suitable chiral phosphine ligand. Palladium acetate is a particularly preferred catalyst. Thus, in any embodiment, the compound XVII can be reacted with Compound II in the presence of potassium carbonate, {(R)-1-[(Sp)-2-(Dicyclohexyl phosphino)ferrocenyl]ethyldi-tert-butylphosphine}[2-(2-amino-1,1-biphenyl)]palladium(II) methanesulfonate (commercial name: Josiphos SL-J009-1-G3-palladacycle), and palladium acetate, in acetonitrile and toluene. The reaction may be carried out at elevated temperature for example at: about 40° C. to about 100° C., about 50° C. to about 90° C., about 60° C. to about 80° C., or about 75° C. The reaction mixture may be cooled, and quenched, for example by the addition of water and aqueous ammonium chloride. The resulting compound XIV may be obtained from the quenched reaction mixture by the addition of an antisolvent, for example toluene. The isolation of compound XIV may be by any suitable method, such as decantation, filtration or centrifuge, preferably by filtration. The solid may be dried, for example under reduced pressure and/or elevated temperature (e.g. about 40° C. to about 100° C., about 50° C. to about 95° C., about 60° C. to about 90° C. or about 70° C. to about 90° C. or about 80° C. Advantageously, the Deucravacitinib can be obtained in high yield as a solid, preferably a crystalline solid, in high yield.

In a further aspect, the above-described process may be generally represented by the below Scheme 2:

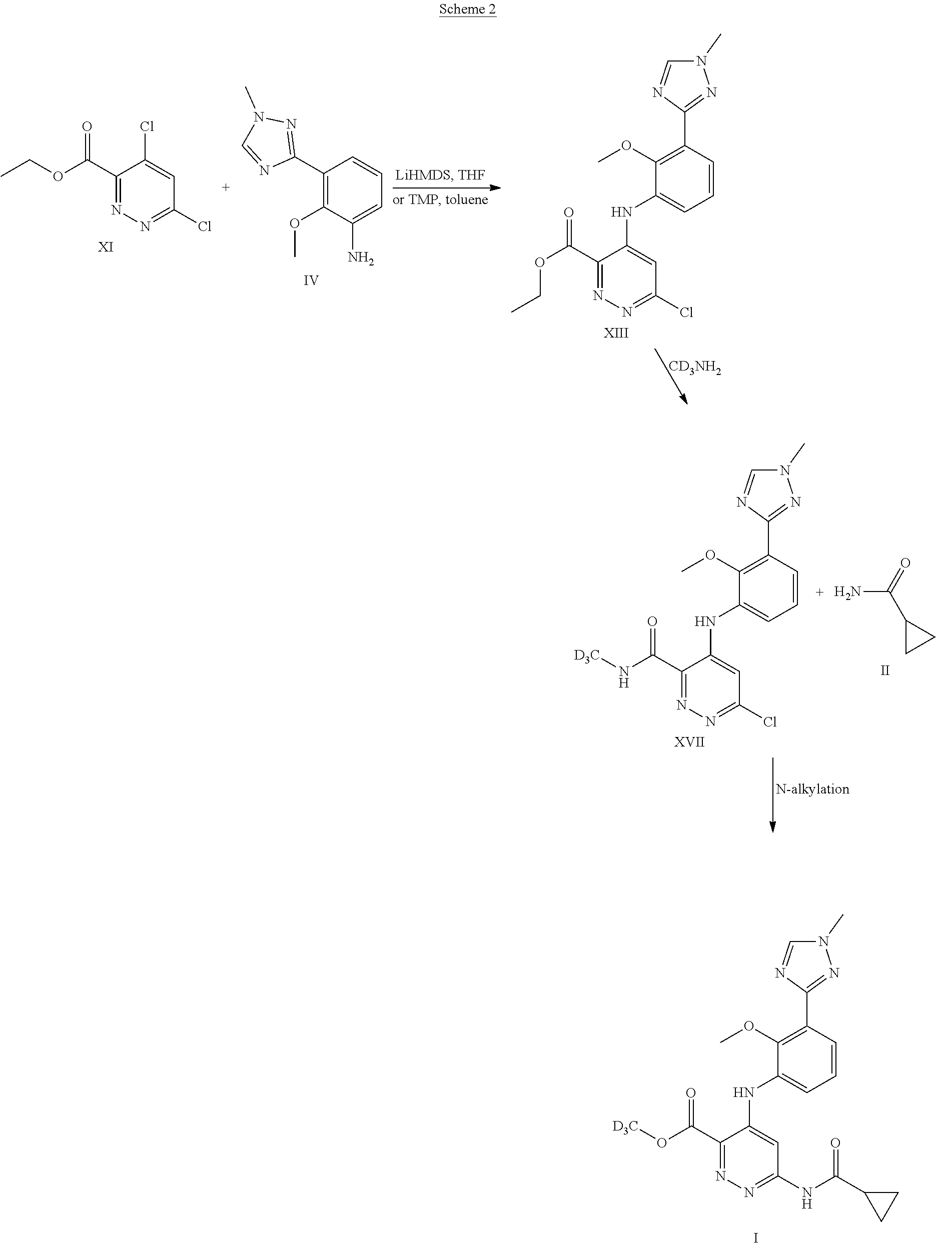

In the above Scheme 2, the reaction of Compound XI with Compound IV may be carried out using lithium bis(trimethylsilyl)amide (LiHMDS) preferably in THE (tetrahydrofuran) solvent, or using 2,2,6,6-tetramethylpiperidine (TMP) preferably in toluene solvent.

As will be appreciated, advantageously in the disclosed processes, deuterated methylamine can react directly with the ethyl ester group of XIII or XIV by aminolysis, and hence the process does not require an additional step of conversion of the ester group to a carboxylic acid or a carboxylic acid salt.

In a specific embodiment, the present disclosure provides Compound XIII:

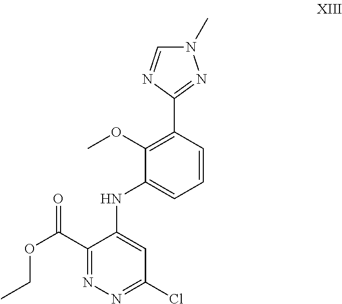 [SMILES: CCOC(=O)c1nnc(Cl)cc1Nc1cccc(-c2ncn(C)n2)c1OC]

### and Compound XIV:

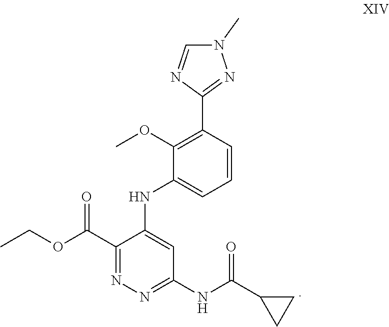 [SMILES: CCOC(=O)c1nnc(NC(=O)C2CC2)cc1Nc1cccc(-c2ncn(C)n2)c1OC]

In another embodiment, the present disclosure provides Compound XVII:

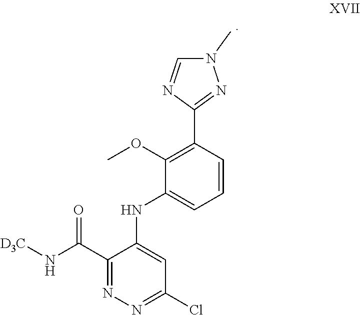 [SMILES: [2H]C([2H])([2H])NC(=O)c1nnc(Cl)cc1Nc1cccc(-c2ncn(C)n2)c1OC]

In another embodiment, the present disclosure provides a process for preparing Deucravacitinib, comprising reacting Compound XVII:

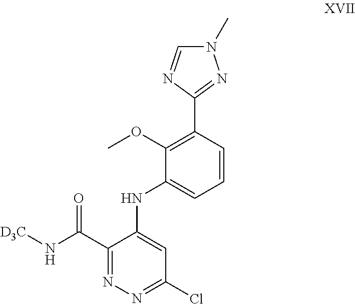 [SMILES: [2H]C([2H])([2H])NC(=O)c1nnc(Cl)cc1Nc1cccc(-c2ncn(C)n2)c1OC]

with cyclopropanecarboxamide in the presence of {(R)-1-[(Sp)-2-(dicyclohexyl phosphino)ferrocenyl]ethyldi-tert-butylphosphine}[2-(2-amino-1,1-biphenyl)]palladium(II) methanesulfonate, Pd(OAc)₂, and an alkali metal base, preferably potassium carbonate; and optionally converting the Deucravacitinib to an acid addition salt thereof. The compound XVII may be prepared by reacting Compound XIII:

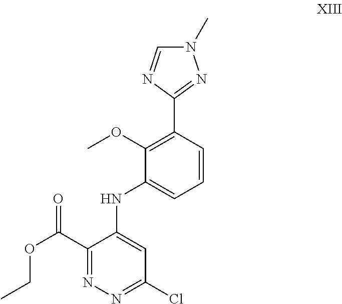 [SMILES: CCOC(=O)c1nnc(Cl)cc1Nc1cccc(-c2ncn(C)n2)c1OC]

with deuterated methylamine or a salt thereof, preferably deuterated methylamine hydrochloride.

Compounds XIII and XIV and XVII may be used as intermediates in preparation of Deucravacitinib. In a specific embodiment, the present disclosure provides the use of Compound XIII and Compound XIV and XVII in the preparation of Deucravacitinib and salts thereof.

Compounds XIII, XIV and XVII are typically isolated, preferably as crystalline solids.

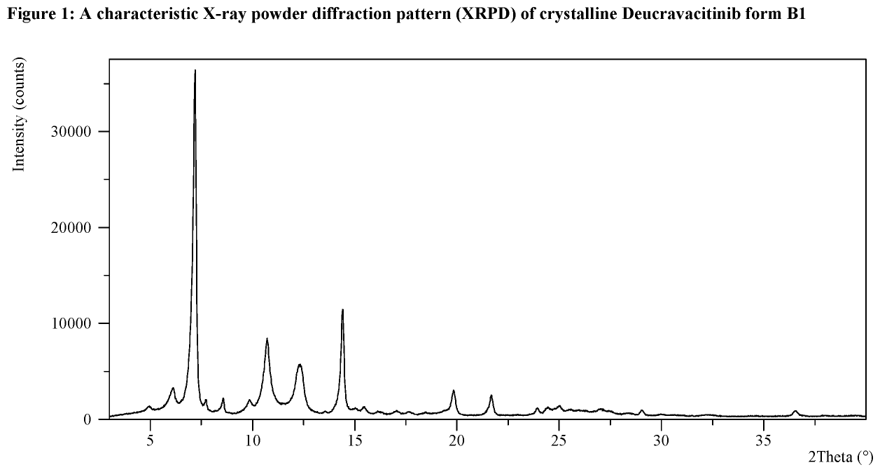

In a specific embodiment, the present disclosure provides crystalline Compound XIII, designated Form A. The crystalline Form A of Compound XIII may be characterized by data selected from one or more of the following: an X-ray powder diffraction pattern substantially as depicted in FIG. 1; an X-ray powder diffraction pattern having peaks at 10.1, 12.6, 14.5, 16.5 and 26.2 degrees 2-theta±0.2 degrees 2-theta; and combinations of these data.

Crystalline Form A of Compound XIII may be further characterized by an X-ray powder diffraction pattern having peaks at 10.1, 12.6, 14.5, 16.5 and 26.2 degrees 2-theta±0.2 degrees 2-theta, and also having any one, two, three, four or five additional peaks selected from 16.9, 19.6, 23.7, 24.4 and 25.5 degrees 2-theta±0.2 degrees 2-theta.

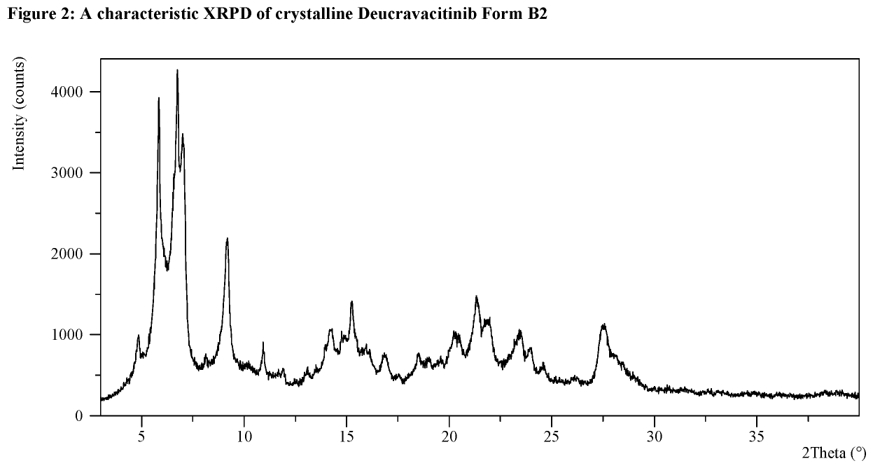

In another specific embodiment, the present disclosure provides crystalline Compound XIV, designated Form 1. The crystalline Form 1 of Compound XIV may be characterized by data selected from one or more of the following: an X-ray powder diffraction pattern substantially as depicted in FIG. 2; an X-ray powder diffraction pattern having peaks at about 6.1, 9.6, 17.9, 21.9, 25.7 degrees 2-theta±0.2 degrees 2-theta; and combinations of these data.

Crystalline Form 1 of Compound XIV may be further characterized by an X-ray powder diffraction pattern having peaks at about 6.1, 9.6, 17.9, 21.9, 25.7 degrees 2-theta±0.2 degrees 2-theta, and also having any one, two, three, four or five additional peaks selected from 12.2, 13.8, 21.1, 22.4 and 24.2 degrees 2-theta±0.2 degrees 2-theta.

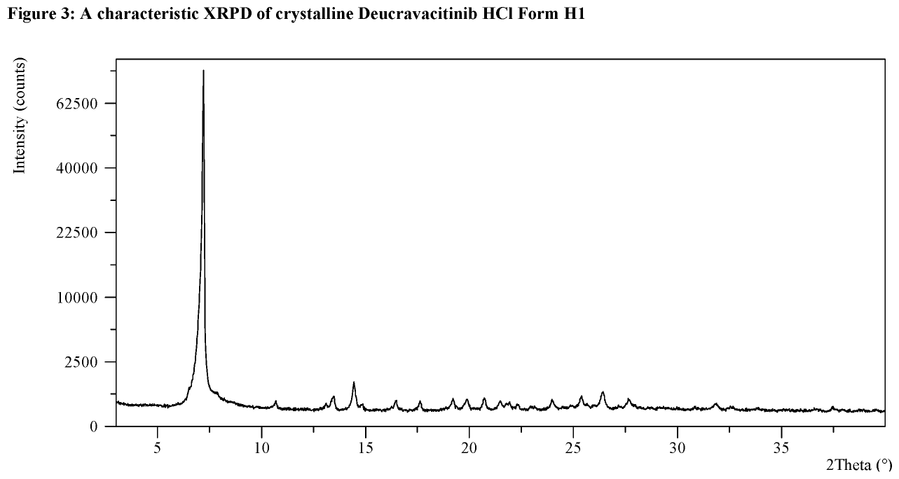

In another specific embodiment, the present disclosure provides crystalline Compound XVII, designated Form B. The crystalline Form B of Compound XVII may be characterized by data selected from one or more of the following: an X-ray powder diffraction pattern substantially as depicted in FIG. 3; an X-ray powder diffraction pattern having peaks at about 7.4, 10.5, 14.9 and 17.5 degrees 2-theta±0.2 degrees 2-theta; and combinations of these data.

Crystalline Form B of Compound XVII may be further characterized by an X-ray powder diffraction pattern having peaks at about 7.4, 10.5, 14.9 and 17.5 degrees 2-theta±0.2 degrees 2-theta, and also having any one, two, three, four or five additional peaks selected from 3.2, 5.3, 13.8, 21.0 and 26.9 degrees 2-theta±0.2 degrees 2-theta.

Compound IV (which is used in both of the above options) can be prepared, in general, as described in the literature, for example as described in WO 2014074661.

In one aspect, Compound IV can be prepared by reduction of the compound of the following formula:

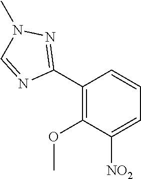 [SMILES: COc1c(-c2ncn(C)n2)cccc1[N+](=O)[O-]]

Alternatively, Compound IV can be prepared from 3-bromo-2-methoxyaniline, by a process that includes activating 3-bromo-2-methoxyaniline, for example by borylation, and reacting the obtained compound with a triazole compound, such as 3-bromo-1-methyl-1H-1,2,4-triazole.

In another aspect, the present disclosure provides the preparation of the intermediate compound, Compound V:

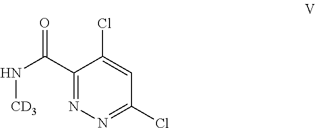 [SMILES: [2H]C([2H])([2H])NC(=O)c1nnc(Cl)cc1Cl]

The process comprises reacting Compound XV:

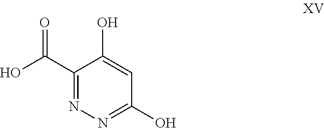 [SMILES: O=C(O)c1nnc(O)cc1O]

with activator and thereafter ammonia, to obtain Compound XVI:

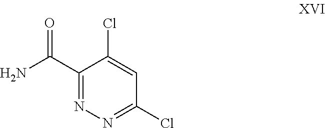 [SMILES: NC(=O)c1nnc(Cl)cc1Cl]

and reacting Compound XVI with deuterated methyl iodide (CDI₃) to obtain Compound V.

An activator can be any compound suitable of forming active groups, such as halogen active groups, in some aspects Cl groups. Suitable activators may be phosphoryl chloride (POCl₃, oxalyl chloride (COCl)₂, thionyl chloride (SOCl₂), etc.

The above-described process is generally represented by the below Scheme 2A:

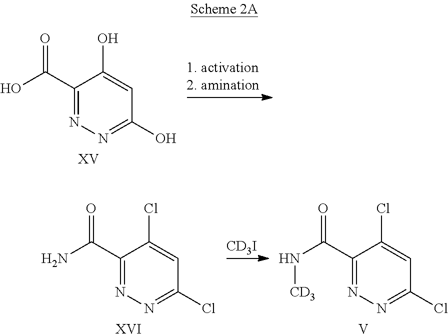

Compound V may be used to prepare Deucravacitinib or Deucravacitinib salt. This process may comprise reacting Compound V with Compound IV, followed by reaction with cyclopropane carboxamide of formula II, for example as described in WO 2014074661.

### Solid State Forms of Deucravacitinib/Deucravacitinib HCl

The present disclosure encompasses solid state forms of Deucravacitinib and of Deucravacitinib HCl, in embodiments the present disclosure encompasses processes for preparation thereof, and pharmaceutical compositions thereof. The present disclosure encompasses Deucravacitinib salts and solid state forms thereof, as well as processes for preparation thereof, and pharmaceutical compositions thereof.

Solid state properties of Deucravacitinib and Deucravacitinib salts and crystalline polymorphs thereof can be influenced by controlling the conditions under which Deucravacitinib and crystalline polymorphs thereof are obtained in solid form.

The solid state form may be referred to herein as “Deucravacitinib Form name” or “Crystalline Form name of Deucravacitinib” or “Crystalline Deucravacitinib Form name” or “Crystalline polymorph name of Deucravacitinib” or “Crystalline Deucravacitinib polymorph name” or “Deucravacitinib polymorph name”. For example, crystalline Form I of Deucravacitinib may be interchangeably referred to herein as Deucravacitinib Form I or as Crystalline Deucravacitinib Form I or as Crystalline polymorph I of Deucravacitinib or as Crystalline Deucravacitinib polymorph I or Deucravacitinib polymorph I.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Deucravacitinib described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Deucravacitinib. In some embodiments of the disclosure, the described crystalline polymorph of Deucravacitinib may contain from about 1% to about 20% (w/w), from about 5% to about 20% (w/w), or from about 5% to about 10% (w/w) of one or more other solid state forms of Deucravacitinib.

Depending on which other crystalline polymorphs a comparison is made, the crystalline polymorphs of Deucravacitinib of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density. For example, Deucravacitinib L-tartaric acid Form It is stable for at least 7 days at exposure to relative humidities of 20%, 40%, 60% and 80%. Advantageously, Deucravacitinib L-tartaric acid Form It is also stable to strong grinding, solvent drop grinding in various solvents, and heating up to 100° C. Deucravacitinib L-tartaric acid Form It also does not convert to another form when exposed to different solvents at ambient temperature. Deucravacitinib L-tartaric acid Form It is also stable to heating at 100° C. for at last 30 minutes. Additionally, Deucravacitinib L-tartaric acid Form It does not convert to another form when compressed (1000 kg and 2000 kg for 1-5 minutes). Deucravacitinib L-tartaric acid Form It also has good solubility in physiological pH values. Deucravacitinib L-tartaric acid Form It is therefore a desirable candidate for formulations.

The present disclosure includes a crystalline polymorph Deucravacitinib designated Form B1. The crystalline Form B1 of Deucravacitinib may be characterized by data selected from one or more of the following: an X-ray powder diffraction pattern substantially as depicted in FIG. 1; an X-ray powder diffraction pattern having peaks at 7.2, 10.7, 19.9 and 21.7 degrees 2-theta±0.2 degrees 2-theta; and combinations of these data.

Crystalline Form B1 of Deucravacitinib may be further characterized by an X-ray powder diffraction pattern having peaks at 7.2, 10.7, 19.9 and 21.7 degrees 2-theta±0.2 degrees 2-theta, and also having any one, two, three, four or five additional peaks selected from 4.9, 6.1, 9.8, 17.0 and 25.0 degrees 2-theta±0.2 degrees 2-theta.

In one embodiment of the present disclosure, crystalline Form B1 of Deucravacitinib is isolated.

Crystalline Form B1 of Deucravacitinib may be anhydrous form. Typically, the water content in B1 is less than 1% (w/w), as detected for example by KF and TGA.

Crystalline Form B1 of Deucravacitinib may be characterized by each of the above characteristics alone/or by all possible combinations, e.g., an XRPD pattern having peaks at 7.2, 10.7, 19.9 and 21.7 degrees 2-theta±0.2 degrees 2-theta; an XRPD pattern as depicted in FIG. 1, and combinations thereof.

The present disclosure includes a crystalline polymorph Deucravacitinib designated Form B2. The crystalline Form B2 of Deucravacitinib may be characterized by data selected from one or more of the following: an X-ray powder diffraction pattern substantially as depicted in FIG. 2; an X-ray powder diffraction pattern having peaks at 5.9, 6.8, 10.9 and 16.8 degrees 2-theta±0.2 degrees 2-theta; and combinations of these data.

Crystalline Form B2 of Deucravacitinib may be further characterized by an X-ray powder diffraction pattern having peaks at 5.9, 6.8, 10.9 and 16.8 degrees 2-theta±0.2 degrees 2-theta, and also having any one, two, three, four or five additional peaks selected from 4.9, 7.1, 9.2, 21.3 and 22.0 degrees 2-theta±0.2 degrees 2-theta.

In one embodiment of the present disclosure, crystalline Form B2 of Deucravacitinib is isolated.

Crystalline Form B2 of Deucravacitinib may be acetic acid solvate-hydrate. Typically, the acetic acid and water total content in Crystalline Form B2 is of about 20% (w/w), as detected for example by KF and TGA.

Crystalline Form B2 of Deucravacitinib may be characterized by each of the above characteristics alone/or by all possible combinations, e.g., an XRPD pattern having peaks at 5.9, 6.8, 10.9 and 16.8 degrees 2-theta±0.2 degrees 2-theta; an XRPD pattern as depicted in FIG. 2, and combinations thereof.

The present disclosure includes a crystalline polymorph Deucravacitinib HCl designated Form H1. The crystalline Form H1 of Deucravacitinib HCl may be characterized by data selected from one or more of the following: an X-ray powder diffraction pattern substantially as depicted in FIG. 3; an X-ray powder diffraction pattern having peaks at 7.2, 14.4, 19.9, 20.7 and 26.4 degrees 2-theta±0.2 degrees 2-theta; and combinations of these data.

Crystalline Form H1 of Deucravacitinib HCl may be further characterized by an X-ray powder diffraction pattern having peaks at 7.2, 14.4, 19.9, 20.7 and 26.4 degrees 2-theta±0.2 degrees 2-theta, and also having any one, two or three additional peaks selected from 10.7, 13.5 and 17.6 degrees 2-theta±0.2 degrees 2-theta.

In one embodiment of the present disclosure, crystalline Form H1 of Deucravacitinib HCl is isolated.

Crystalline Form H1 of Deucravacitinib HCl may be a solvate, typically an acetonitrile solvate. Typically, the acetonitrile content in Crystalline Form H1 is of about 7.2% (w/w), as detected for example by KF and TGA.

Crystalline Form H1 of Deucravacitinib HCl may be characterized by each of the above characteristics alone/or by all possible combinations, e.g., an XRPD pattern having peaks at 7.2, 14.4, 19.9, 20.7 and 26.4 degrees 2-theta±0.2 degrees 2-theta; an XRPD pattern as depicted in FIG. 3, and combinations thereof.

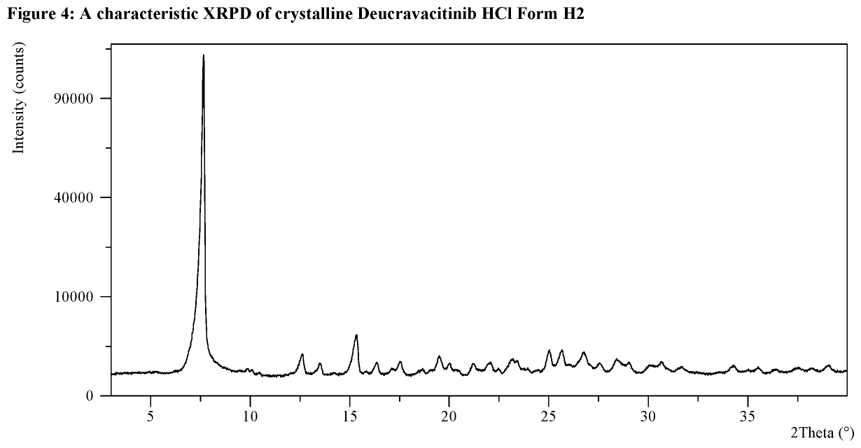

The present disclosure includes a crystalline polymorph Deucravacitinib HCl designated Form H2. The crystalline Form H2 of Deucravacitinib HCl may be characterized by data selected from one or more of the following: an X-ray powder diffraction pattern substantially as depicted in FIG. 4; an X-ray powder diffraction pattern having peaks at 7.7, 13.5, 17.6 and 25.7 degrees 2-theta±0.2 degrees 2-theta; and combinations of these data.

Crystalline Form H2 of Deucravacitinib HCl may be further characterized by an X-ray powder diffraction pattern having peaks at 7.7, 13.5, 17.6 and 25.7 degrees 2-theta±0.2 degrees 2-theta, and also having any one, two or three additional peaks selected from 19.5, 20.0 and 25.0 degrees 2-theta±0.2 degrees 2-theta.

In one embodiment of the present disclosure, crystalline Form H2 of Deucravacitinib HCl is isolated.

Crystalline Form H2 of Deucravacitinib HCl may be an anhydrous form. Typically, the water content in Crystalline Form H2 of Deucravacitinib HCl is less than 1% (w/w), as detected for example by KF and TGA.

Crystalline Form H2 of Deucravacitinib HCl may be characterized by each of the above characteristics alone/or by all possible combinations, e.g., an XRPD pattern having peaks at 7.7, 13.5, 17.6 and 25.7 degrees 2-theta±0.2 degrees 2-theta; an XRPD pattern as depicted in FIG. 4, and combinations thereof.

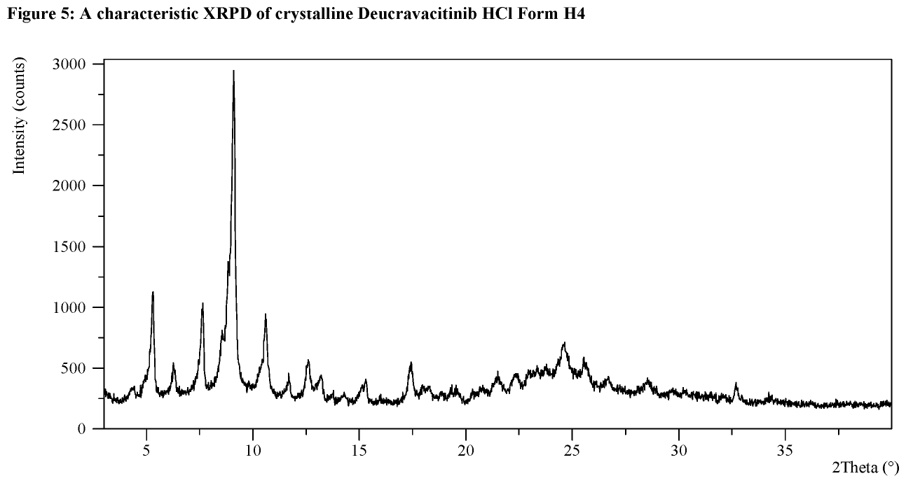

The present disclosure includes a crystalline polymorph Deucravacitinib HCl designated Form H4. The crystalline Form H4 of Deucravacitinib HCl may be characterized by data selected from one or more of the following: an X-ray powder diffraction pattern substantially as depicted in FIG. 5; an X-ray powder diffraction pattern having peaks at 5.3, 6.3, 7.7, 9.1 and 17.4 degrees 2-theta±0.2 degrees 2-theta; and combinations of these data.

Crystalline Form H4 of Deucravacitinib HCl may be further characterized by an X-ray powder diffraction pattern having peaks at 5.3, 6.3, 7.7, 9.1 and 17.4 degrees 2-theta±0.2 degrees 2-theta, and also having any one, two or three additional peaks selected from 11.7, 13.2, 24.6 degrees 2-theta±0.2 degrees 2-theta.

In one embodiment of the present disclosure, crystalline Form H4 of Deucravacitinib HCl is isolated.

Crystalline Form H4 of Deucravacitinib HCl may be an anhydrous form or a hydrate form. Typically, the water content in Crystalline Form H4 of Deucravacitinib HCl is of up to 3.8%, as detected, for example, by TGA, at a temperature of up to 100° C. (w/w, i.e. weight loss by TGA).

Crystalline Form H4 of Deucravacitinib HCl may be characterized by each of the above characteristics alone/or by all possible combinations, e.g., an XRPD pattern having peaks at 5.3, 6.3, 7.7, 9.1 and 17.4 degrees 2-theta±0.2 degrees 2-theta; an XRPD pattern as depicted in FIG. 5, and combinations thereof.

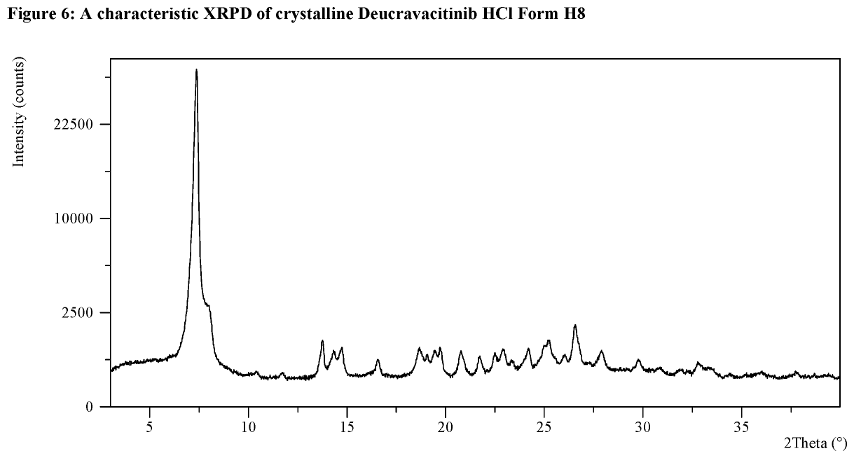

The present disclosure includes a crystalline polymorph Deucravacitinib HCl designated Form H8. The crystalline Form H8 of Deucravacitinib HCl may be characterized by data selected from one or more of the following: an X-ray powder diffraction pattern substantially as depicted in FIG. 6; an X-ray powder diffraction pattern having peaks at 7.4, 13.8, 14.8, 21.7 and 26.6 degrees 2-theta±0.2 degrees 2-theta; and combinations of these data.

Crystalline Form H8 of Deucravacitinib HCl may be further characterized by an X-ray powder diffraction pattern having peaks at 7.4, 13.8, 14.8, 21.7 and 26.6 degrees 2-theta 0.2 degrees 2-theta, and also having additional peak selected at 24.2 degrees 2-theta±0.2 degrees 2-theta.

In one embodiment of the present disclosure, crystalline Form H8 of Deucravacitinib HCl is isolated.

Crystalline Form H8 of Deucravacitinib HCl may be a solvate form, preferably an acetic acid solvate.

Crystalline Form H8 of Deucravacitinib HCl may be characterized by each of the above characteristics alone/or by all possible combinations, e.g., an XRPD pattern having peaks at 7.4, 13.8, 14.8, 21.7 and 26.6 degrees 2-theta±0.2 degrees 2-theta; an XRPD pattern as depicted in FIG. 6, and combinations thereof.

The present disclosure further encompasses crystalline Deucravacitinib HCl:L-Malic acid. Crystalline Deucravacitinib HCl:L-Malic acid may be a co-crystal of Deucravacitinib HCl and L-Malic acid. Alternatively, crystalline Deucravacitinib HCl:L-Malic acid may be a salt, i.e., Deucravacitinib HCl-Malate.

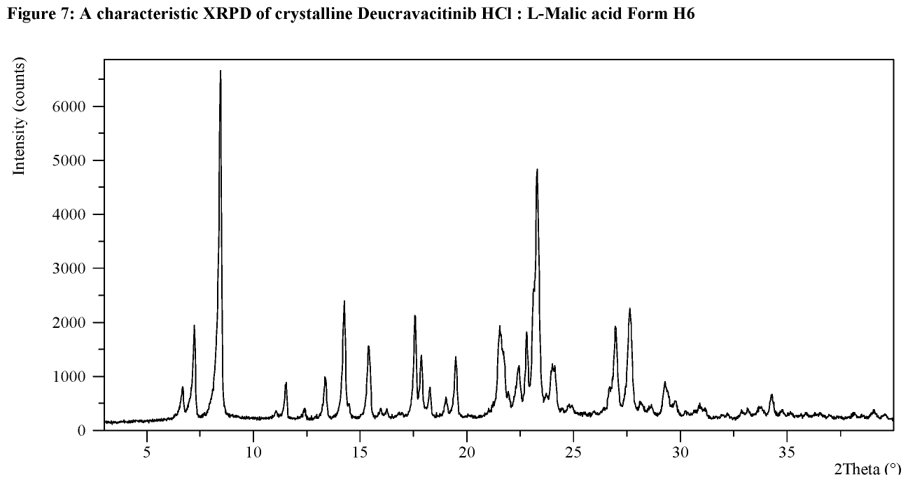

The disclosure further encompasses a crystalline form of Deucravacitinib HCl:L-Malic acid, designated form H6. Crystalline Form H6 of Deucravacitinib HCl:L-Malic acid may be characterized by data selected from one or more of the following: an X-ray powder diffraction pattern substantially as depicted in FIG. 7; an X-ray powder diffraction pattern having peaks at 6.7, 7.2, 8.5, 14.3 and 23.3 degrees 2-theta±0.2 degrees 2-theta; and combinations of these data.

Crystalline Form H6 of Deucravacitinib HCl:L-Malic acid may be further characterized by an X-ray powder diffraction pattern having peaks at 6.7, 7.2, 8.5, 14.3 and 23.3 degrees 2-theta±0.2 degrees 2-theta, and also having any one, two, three, four or five additional peaks selected from 11.5, 13.4, 17.6, 17.9 and 27.7 degrees 2-theta±0.2 degrees 2-theta.

In embodiments of the present disclosure, crystalline Form H6 of Deucravacitinib HCl:L-Malic acid is isolated.

Crystalline Form H6 of Deucravacitinib HCl:L-Malic acid may be an anhydrous form.

Crystalline Form H6 of Deucravacitinib HCl:L-Malic acid may be characterized by each of the above characteristics alone or by all possible combinations, e.g., an XRPD pattern having peaks at 6.7, 7.2, 8.5, 14.3 and 23.3 degrees 2-theta±0.2 degrees 2-theta; an XRPD pattern as depicted in FIG. 7; and combinations thereof.

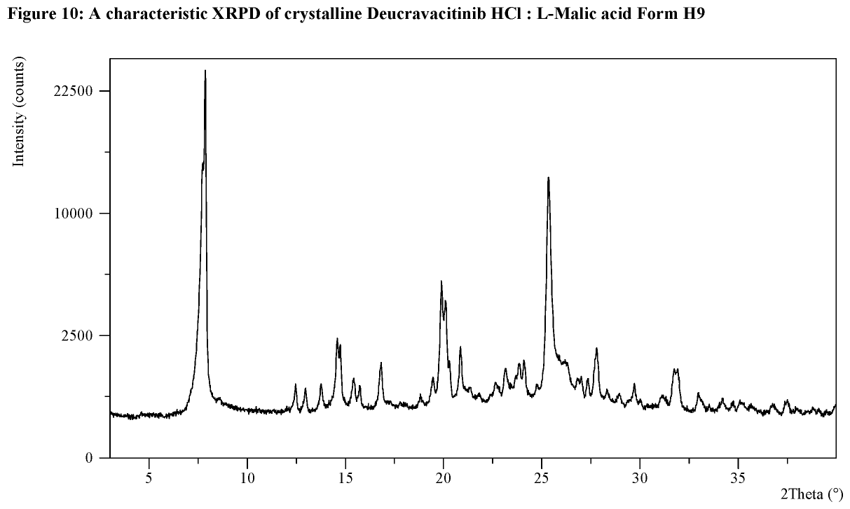

The disclosure further encompasses a crystalline form of Deucravacitinib HCl:L-Malic acid, designated form H9. Crystalline Form H9 of Deucravacitinib HCl:L-Malic acid may be characterized by data selected from one or more of the following: an X-ray powder diffraction pattern substantially as depicted in FIG. 10; an X-ray powder diffraction pattern having peaks at 7.9, 16.8, 19.9 and 25.3 degrees 2-theta 0.2 degrees 2-theta; and combinations of these data.

Crystalline Form H9 of Deucravacitinib HCl:L-Malic acid may be further characterized by an X-ray powder diffraction pattern having peaks at 7.9, 16.8, 19.9 and 25.3 degrees 2-theta±0.2 degrees 2-theta, and also having any one, two, three or four additional peaks selected from 12.5, 13.0, 13.8 and 14.6 degrees 2-theta±0.2 degrees 2-theta.

In embodiments of the present disclosure, crystalline Form H9 of Deucravacitinib HCl:L-Malic acid is isolated.

Crystalline Form H9 of Deucravacitinib HCl:L-Malic acid may be characterized by each of the above characteristics alone or by all possible combinations, e.g., an XRPD pattern having peaks at 7.9, 16.8, 19.9 and 25.3 degrees 2-theta±0.2 degrees 2-theta; an XRPD pattern as depicted in FIG. 10; and combinations thereof.

The present disclosure further encompasses crystalline Deucravacitinib:L-Malic acid, i.e. Deucravacitinib free base and L-Malic complex. Crystalline Deucravacitinib:L-Malic acid may be a co-crystal of Deucravacitinib and L-Malic acid. Alternatively, crystalline Deucravacitinib:L-Malic acid may be a salt, i.e., Deucravacitinib L-Malate.

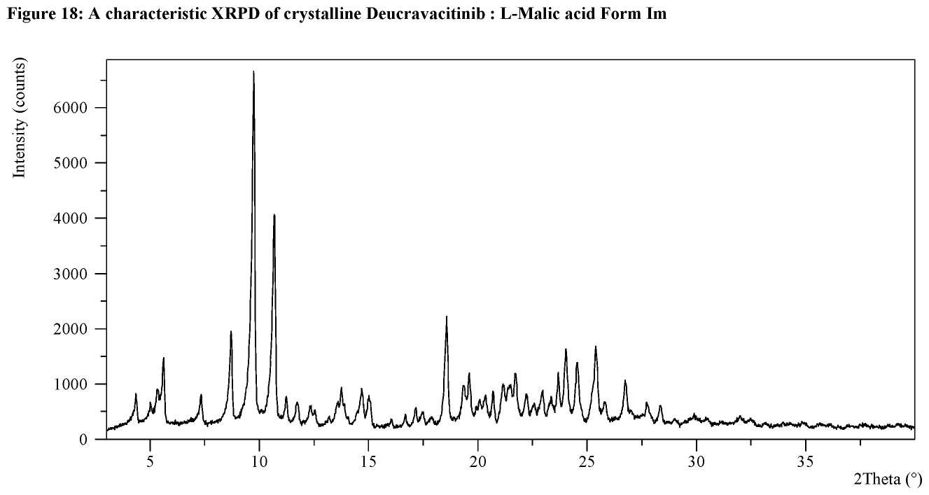

The disclosure further encompasses a crystalline form of Deucravacitinib:L-Malic acid, designated form Im. Crystalline Form Im of Deucravacitinib:L-Malic acid may be characterized by data selected from one or more of the following: an X-ray powder diffraction pattern substantially as depicted in FIG. 18; an X-ray powder diffraction pattern having peaks at 9.8, 10.7, 18.6 and 19.6 degrees 2-theta±0.2 degrees 2-theta; and combinations of these data.

Crystalline Form Im of Deucravacitinib:L-Malic acid may be further characterized by an X-ray powder diffraction pattern having peaks at 9.8, 10.7, 18.6 and 19.6 degrees 2-theta±0.2 degrees 2-theta, and also having any one, two, three or four additional peaks selected from 4.4, 14.7, 24.0 and 24.5 degrees 2-theta±0.2 degrees 2-theta.

In embodiments of the present disclosure, crystalline Form Im of Deucravacitinib:L-Malic acid is isolated.

Crystalline Form Im of Deucravacitinib:L-Malic acid may be an anhydrous form.

Crystalline Form Im of Deucravacitinib:L-Malic acid may be characterized by each of the above characteristics alone or by all possible combinations, e.g., an XRPD pattern having peaks at 9.8, 10.7, 18.6 and 19.6 degrees 2-theta±0.2 degrees 2-theta; an XRPD pattern as depicted in FIG. 18; and combinations thereof.

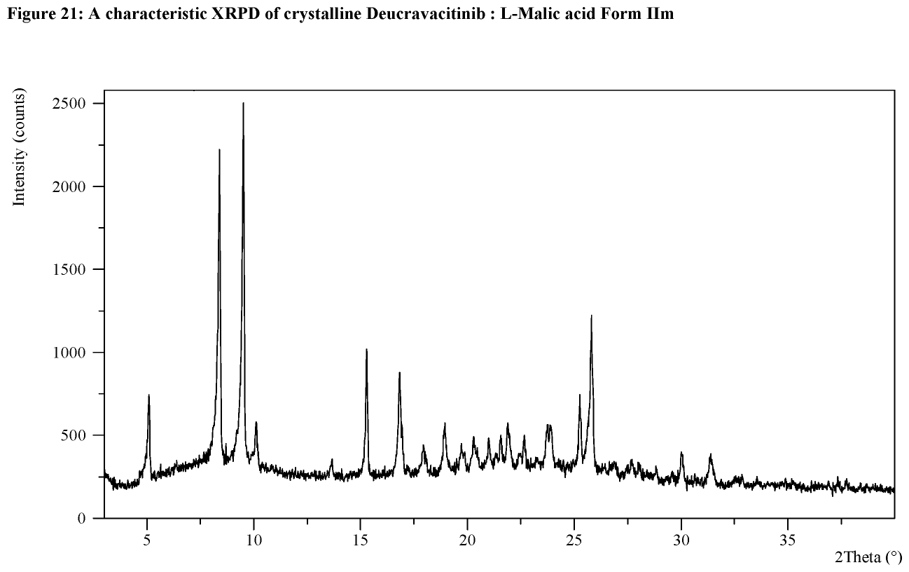

The disclosure also encompasses a crystalline form of Deucravacitinib:L-Malic acid, designated form IIm. Crystalline Form IIm of Deucravacitinib:L-Malic acid may be characterized by data selected from one or more of the following: an X-ray powder diffraction pattern substantially as depicted in FIG. 21; an X-ray powder diffraction pattern having peaks at 5.1, 9.5, 15.3 and 16.8 degrees 2-theta±0.2 degrees 2-theta; and combinations of these data.

Crystalline Form IIm of Deucravacitinib:L-Malic acid may be further characterized by an X-ray powder diffraction pattern having peaks at 5.1, 9.5, 15.3 and 16.8 degrees 2-theta 0.2 degrees 2-theta, and also having any one, two, three or four additional peaks selected from 8.4, 17.9, 21.9 and 25.8 degrees 2-theta±0.2 degrees 2-theta.

In embodiments of the present disclosure, crystalline Form IIm of Deucravacitinib:L-Malic acid is isolated.

Crystalline Form IIm of Deucravacitinib:L-Malic acid may be a solvate-hydrate form.

Crystalline Form IIm of Deucravacitinib:L-Malic acid may be characterized by each of the above characteristics alone or by all possible combinations, e.g., an XRPD pattern having peaks at 5.1, 9.5, 15.3 and 16.8 degrees 2-theta±0.2 degrees 2-theta; an XRPD pattern as depicted in FIG. 21; and combinations thereof.

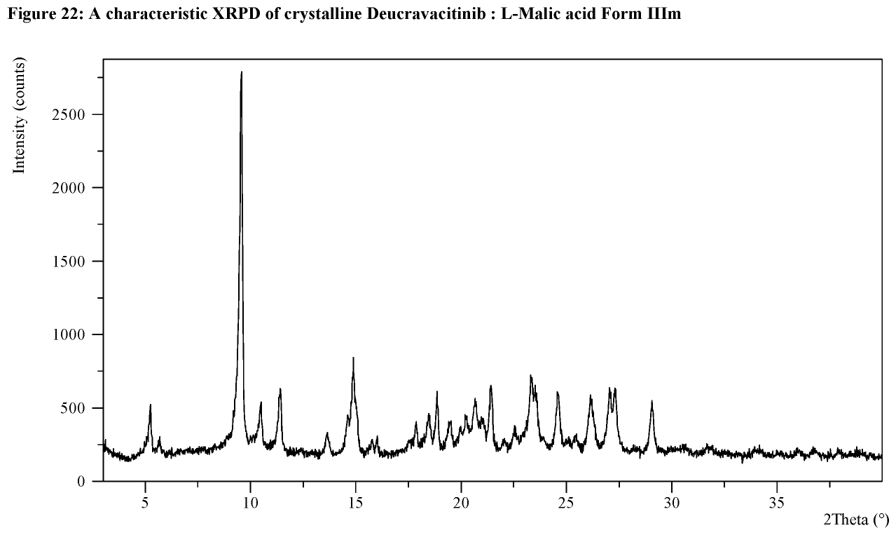

The disclosure also encompasses a crystalline form of Deucravacitinib:L-Malic acid, designated form IIIm. Crystalline Form IIIm of Deucravacitinib:L-Malic acid may be characterized by data selected from one or more of the following: an X-ray powder diffraction pattern substantially as depicted in FIG. 22; an X-ray powder diffraction pattern having peaks at 9.6, 10.5, 14.9, 24.6 and 26.1 degrees 2-theta±0.2 degrees 2-theta; and combinations of these data.

Crystalline Form IIIm of Deucravacitinib:L-Malic acid may be further characterized by an X-ray powder diffraction pattern having peaks at 9.6, 10.5, 14.9, 24.6 and 26.1 degrees 2-theta±0.2 degrees 2-theta, and also having any one, two, three or four additional peaks selected from 5.2, 13.6, 17.9 and 18.5 degrees 2-theta±0.2 degrees 2-theta.

In embodiments of the present disclosure, crystalline Form IIIm of Deucravacitinib: L-Malic acid is isolated.

Crystalline Form IIIm of Deucravacitinib:L-Malic acid may be an anhydrous form.

Crystalline Form IIIm of Deucravacitinib:L-Malic acid may be characterized by each of the above characteristics alone or by all possible combinations, e.g., an XRPD pattern having peaks at 9.6, 10.5, 14.9, 24.6 and 26.1 degrees 2-theta±0.2 degrees 2-theta; an XRPD pattern as depicted in FIG. 22; and combinations thereof.

The present disclosure further encompasses crystalline Deucravacitinib HCl:Urea. Crystalline Deucravacitinib HCl:Urea acid may be a co-crystal of Deucravacitinib HCl and Urea.

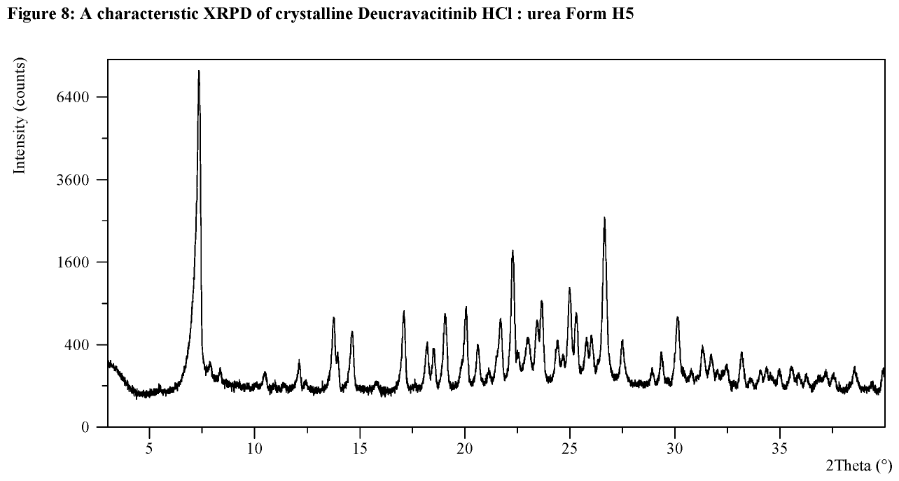

The disclosure further encompasses a crystalline form of Deucravacitinib HCl:Urea, designated form H5. Crystalline Form H5 of Deucravacitinib HCl:Urea may be characterized by data selected from one or more of the following: an X-ray powder diffraction pattern substantially as depicted in FIG. 8; an X-ray powder diffraction pattern having peaks at 7.3, 17.1, 20.1, 21.7 and 26.6 degrees 2-theta±0.2 degrees 2-theta; and combinations of these data.

Crystalline Form H5 of Deucravacitinib HCl:Urea may be further characterized by an X-ray powder diffraction pattern having peaks at 7.3, 17.1, 20.1, 21.7 and 26.6 degrees 2-theta±0.2 degrees 2-theta, and also having any one, two, three, four or five additional peaks selected from 14.7, 18.2, 20.6, 25.0 and 27.5 degrees 2-theta±0.2 degrees 2-theta.

In embodiments of the present disclosure, crystalline Form H5 of Deucravacitinib HCl:Urea is isolated.

Crystalline Form H5 of Deucravacitinib HCl:Urea may be an anhydrous form.

Crystalline Form H5 of Deucravacitinib HCl:Urea may be characterized by each of the above characteristics alone or by all possible combinations, e.g., an XRPD pattern having peaks at 7.3, 17.1, 20.1, 21.7 and 26.6 degrees 2-theta±0.2 degrees 2-theta; an XRPD pattern as depicted in FIG. 8; and combinations thereof.

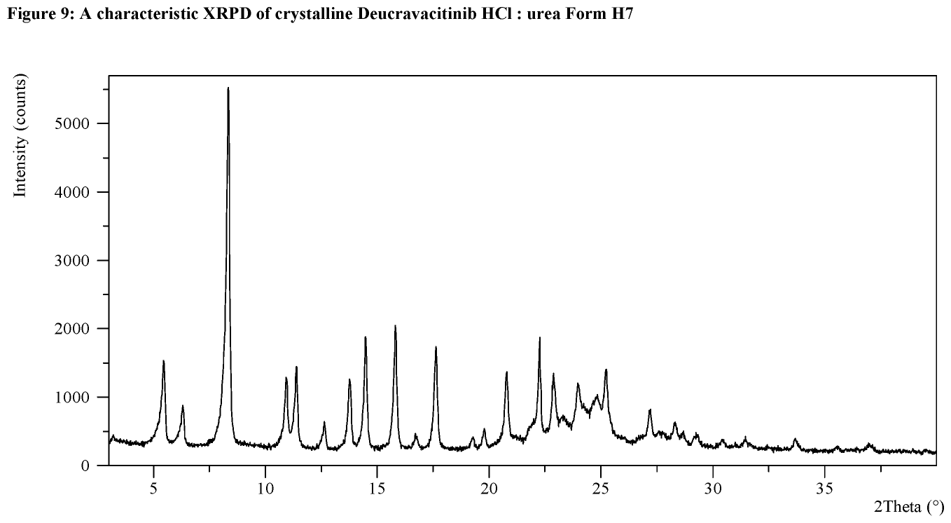

The disclosure further encompasses a crystalline form of Deucravacitinib HCl:Urea, designated form H7. Crystalline Form H7 of Deucravacitinib HCl:Urea may be characterized by data selected from one or more of the following: an X-ray powder diffraction pattern substantially as depicted in FIG. 9; an X-ray powder diffraction pattern having peaks at 5.5, 8.4, 10.9, 11.4 and 14.5 degrees 2-theta±0.2 degrees 2-theta; and combinations of these data.

Crystalline Form H7 of Deucravacitinib HCl:Urea may be further characterized by an X-ray powder diffraction pattern having peaks at 5.5, 8.4, 10.9, 11.4 and 14.5 degrees 2-theta±0.2 degrees 2-theta, and also having any one, two, three, four or five additional peaks selected from 6.3, 13.8, 15.8, 17.6 and 22.3 degrees 2-theta±0.2 degrees 2-theta.

In embodiments of the present disclosure, crystalline Form H5 of Deucravacitinib HCl:Urea is isolated.

Crystalline Form H7 of Deucravacitinib HCl:Urea may be an anhydrous form.

Crystalline Form H7 of Deucravacitinib HCl:Urea may be characterized by each of the above characteristics alone or by all possible combinations, e.g., an XRPD pattern having peaks at 5.5, 8.4, 10.9, 11.4 and 14.5 degrees 2-theta±0.2 degrees 2-theta; an XRPD pattern as depicted in FIG. 9; and combinations thereof.

The disclosure further encompasses Deucravacitinib salts and their solid state forms thereof, particularly crystalline forms. In embodiments, the present disclosure encompasses Deucravacitinib HBr, Deucravacitinib edisylate (“EDSA” or ethanedisulfonate) salt, Deucravacitinib esylate (“ESA” or ethanesulfonate) salt and their solid state forms, particularly crystalline forms.

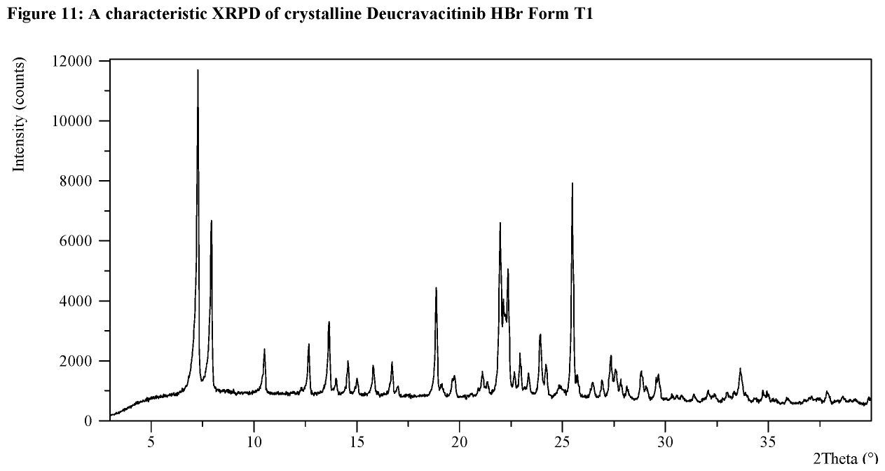

The disclosure further encompasses a crystalline form of Deucravacitinib HBr, designated form T1. Crystalline Form T1 of Deucravacitinib HBr may be characterized by data selected from one or more of the following: an X-ray powder diffraction pattern substantially as depicted in FIG. 11; an X-ray powder diffraction pattern having peaks at 7.3, 7.9, 13.7, 22.0 and 23.9 degrees 2-theta±0.2 degrees 2-theta; and combinations of these data.

Crystalline Form T1 of Deucravacitinib HBr may be further characterized by an X-ray powder diffraction pattern having peaks at 7.3, 7.9, 13.7, 22.0 and 23.9 degrees 2-theta±0.2 degrees 2-theta, and also having any one, two, three, four or five additional peaks selected from 15.0, 16.7, 22.9, 24.2 and 25.5 degrees 2-theta±0.2 degrees 2-theta.

Crystalline Form T1 of Deucravacitinib HBr may be characterized by an X-ray powder diffraction pattern having peaks as listed in Table 1. Optionally, Crystalline Form T1 of Deucravacitinib HBr may be characterized by an XRPD pattern having the 2-theta values in Table 1 together with an error value of ±0.2 degrees 2-theta, either with or without the respective relative intensity values):

TABLE 1

| Pos. [°2Th.] | Rel. Int. [%] |
|---|---|
| 7.3 | 100 |
| 7.9 | 56.14 |
| 10.5 | 14.66 |
| 12.7 | 16.5 |
| 13.7 | 23.44 |
| 14.0 | 5.53 |
| 14.6 | 10.95 |
| 15.0 | 5.68 |
| 15.8 | 10.15 |
| 16.7 | 11.22 |
| 18.9 | 34.2 |
| 19.6 | 6.2 |
| 19.8 | 7.21 |
| 21.1 | 7.76 |
| 21.3 | 5.45 |
| 22.0 | 55.5 |
| 22.1 | 30.64 |
| 22.4 | 40.13 |
| 22.7 | 8.66 |
| 22.9 | 13.46 |
| 23.3 | 8.23 |
| 23.9 | 20.62 |
| 24.2 | 10.94 |
| 25.5 | 67.27 |
| 25.7 | 7.84 |
| 26.5 | 5.56 |
| 26.9 | 6.13 |
| 27.3 | 14.11 |
| 27.6 | 9.68 |
| 27.8 | 6.09 |
| 28.8 | 8.82 |
| 29.5 | 6.99 |
| 29.7 | 8.54 |
| 33.6 | 10.62 |

In embodiments of the present disclosure, crystalline Form T1 of Deucravacitinib HBr is isolated.

Crystalline Form T1 of Deucravacitinib HBr may be a hydrate form. The water content may of from about 6% to 7.5%, for example 6.78% (w/w) as determined by Karl-Fischer.

Crystalline Form T1 of Deucravacitinib HBr may be characterized by each of the above characteristics alone or by all possible combinations, e.g., an XRPD pattern having peaks at 7.3, 7.9, 13.7, 22.0 and 23.9 degrees 2-theta±0.2 degrees 2-theta; an XRPD pattern as depicted in FIG. 11; and combinations thereof.

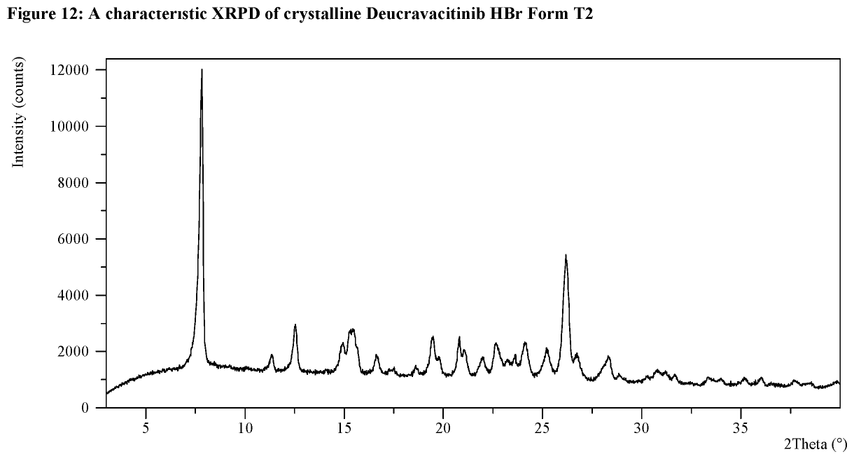

The disclosure further encompasses a crystalline form of Deucravacitinib HBr, designated form T2. Crystalline Form T2 of Deucravacitinib HBr may be characterized by data selected from one or more of the following: an X-ray powder diffraction pattern substantially as depicted in FIG. 12; an X-ray powder diffraction pattern having peaks at 7.8, 11.4, 20.8, 22.6 and 26.2 degrees 2-theta±0.2 degrees 2-theta; and combinations of these data.

Crystalline Form T2 of Deucravacitinib HBr may be further characterized by an X-ray powder diffraction pattern having peaks at 7.8, 11.4, 20.8, 22.6 and 26.2 degrees 2-theta±0.2 degrees 2-theta, and also having any one, two or three additional peaks selected from 16.6, 22.0 and 24.2 degrees 2-theta±0.2 degrees 2-theta.

Crystalline Form T2 of Deucravacitinib HBr may be characterized by an X-ray powder diffraction pattern having peaks as listed in Table 2. Optionally, Crystalline Form T2 of Deucravacitinib HBr may be characterized by an XRPD pattern having the 2-theta values in Table 2 together with an error value of ±0.2 degrees 2-theta, either with or without the respective relative intensity values):

TABLE 2

| Pos. [°2Th.] | Rel. Int. [% ] |
|---|---|
| 7.8 | 100 |
| 12.5 | 15.57 |
| 14.9 | 10.59 |
| 15.2 | 14.16 |
| 15.5 | 14.25 |
| 15.7 | 8.92 |
| 16.6 | 6.63 |
| 19.4 | 12.16 |
| 19.5 | 13.03 |
| 19.8 | 6.67 |
| 20.8 | 13.24 |
| 21.1 | 8.88 |
| 22.0 | 6.79 |
| 22.6 | 11.26 |
| 23.2 | 6.21 |
| 23.6 | 8.23 |
| 24.2 | 12.43 |
| 25.2 | 10.54 |
| 26.1 | 30.86 |
| 26.2 | 41.5 |
| 26.8 | 8.47 |
| 28.3 | 8.18 |

In embodiments of the present disclosure, crystalline Form T2 of Deucravacitinib HBr is isolated.

Crystalline Form T2 of Deucravacitinib HBr may be an anhydrous form.

Crystalline Form T2 of Deucravacitinib HBr may be characterized by each of the above characteristics alone or by all possible combinations, e.g., an XRPD pattern having peaks at 7.8, 11.4, 20.8, 22.6 and 26.2 degrees 2-theta±0.2 degrees 2-theta; an XRPD pattern as depicted in FIG. 12; and combinations thereof.

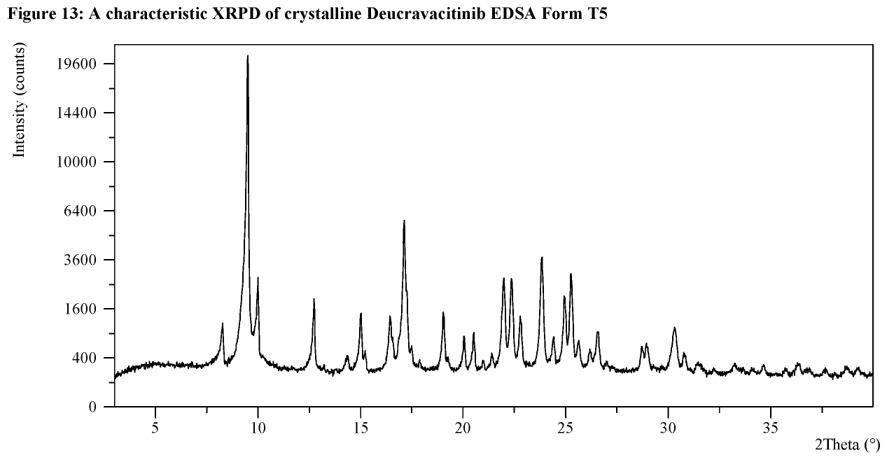

The disclosure further encompasses a crystalline form of Deucravacitinib ethanedisulfonate (“EDSA” or edisylate), designated form T5. Crystalline Form T5 of Deucravacitinib EDSA may be characterized by data selected from one or more of the following: an X-ray powder diffraction pattern substantially as depicted in FIG. 13; an X-ray powder diffraction pattern having peaks at 9.5, 12.7, 17.1, 22.0 and 22.8 degrees 2-theta±0.2 degrees 2-theta; and combinations of these data.

Crystalline Form T5 of Deucravacitinib EDSA may be further characterized by an X-ray powder diffraction pattern having peaks at 9.5, 12.7, 17.1, 22.0 and 22.8 degrees 2-theta±0.2 degrees 2-theta, and also having any one, two, three or four additional peaks selected from 8.3, 16.4, 23.8 and 24.9 degrees 2-theta±0.2 degrees 2-theta.

Crystalline Form T5 of Deucravacitinib EDSA may be characterized by an X-ray powder diffraction pattern having peaks as listed in Table 3. Optionally, Crystalline Form T5 of Deucravacitinib EDSA may be characterized by an XRPD pattern having the 2-theta values in Table 3 with an error value of ±0.2 degrees 2-theta, either with or without the respective relative intensity values):

TABLE 3

| Pos. [°2Th.] | Rel. Int. [% ] |
|---|---|
| 9.5 | 100 |
| 10.0 | 12.43 |
| 12.7 | 8.4 |
| 15.0 | 6.25 |
| 16.4 | 5.83 |
| 17.1 | 27.43 |
| 17.3 | 8.84 |
| 19.0 | 6.09 |
| 21.4 | 1.26 |
| 22.0 | 12.85 |
| 22.4 | 12.58 |
| 22.8 | 5.52 |
| 23.8 | 17.66 |
| 24.9 | 9.35 |
| 25.3 | 13.75 |

In embodiments of the present disclosure, crystalline Form T5 of Deucravacitinib EDSA is isolated.

Crystalline Form T5 of Deucravacitinib EDSA may be an anhydrous form.

Crystalline Form T5 of Deucravacitinib EDSA may be characterized by each of the above characteristics alone or by all possible combinations, e.g., an XRPD pattern having peaks at 9.5, 12.7, 17.1, 22.0 and 22.8 degrees 2-theta±0.2 degrees 2-theta; an XRPD pattern as depicted in FIG. 13; and combinations thereof.

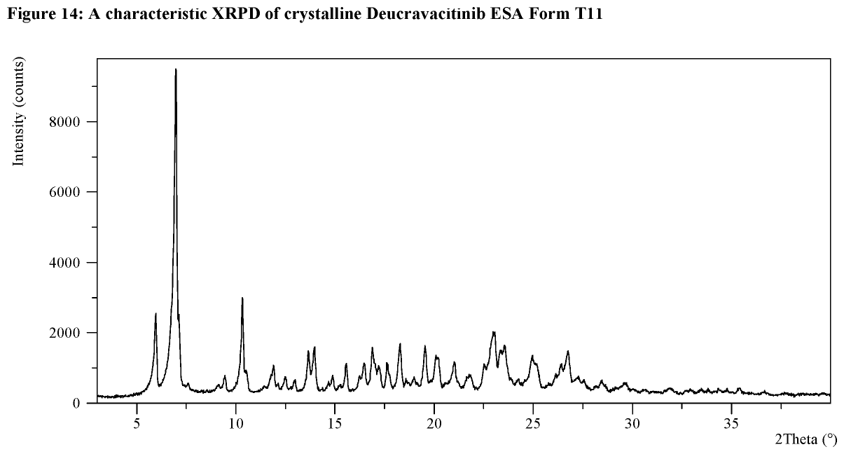

The disclosure further encompasses a crystalline form of Deucravacitinib ethanesulfonate (“ESA” or esylate), designated form T11. Crystalline Form T11 of Deucravacitinib ESA may be characterized by data selected from one or more of the following: an X-ray powder diffraction pattern substantially as depicted in FIG. 14; an X-ray powder diffraction pattern having peaks at 6.0, 7.0, 18.3, 19.5 and 21.0 degrees 2-theta±0.2 degrees 2-theta; and combinations of these data.

Crystalline Form T11 of Deucravacitinib ESA may be further characterized by an X-ray powder diffraction pattern having peaks at 6.0, 7.0, 18.3, 19.5 and 21.0 degrees 2-theta±0.2 degrees 2-theta, and also having any one, two, three, four or five additional peaks selected from 14.0, 15.6, 16.5, 16.9 and 17.6 degrees 2-theta±0.2 degrees 2-theta.

Crystalline Form T11 of Deucravacitinib ESA may be characterized by an X-ray powder diffraction pattern having peaks as listed in Table 4. Crystalline Form T11 of Deucravacitinib ESA may be characterized by an XRPD pattern having the 2-theta values in Table 4 with an error value of ±0.2 degrees 2-theta, either with or without the respective relative intensity values):

TABLE 4

| Pos. [°2Th.] | Rel. Int. [%] |
|---|---|
| 6.0 | 25.15 |
| 7.0 | 100 |
| 7.2 | 22.81 |
| 9.5 | 6.01 |
| 10.3 | 29.58 |
| 10.6 | 6.38 |
| 11.9 | 9.33 |
| 12.5 | 5.74 |
| 13.7 | 13.59 |
| 14.0 | 14.95 |
| 14.9 | 6.17 |
| 15.6 | 9.79 |
| 16.2 | 5.82 |
| 16.5 | 9.56 |
| 16.9 | 14.57 |
| 17.2 | 8.52 |
| 17.6 | 10.09 |
| 18.3 | 15.61 |
| 19.0 | 5.65 |
| 19.5 | 15.25 |
| 20.1 | 12.04 |
| 20.2 | 11.02 |
| 21.0 | 10.16 |
| 21.9 | 6.07 |
| 22.5 | 9.67 |
| 23.1 | 19.17 |
| 23.3 | 13.29 |
| 23.6 | 14.96 |
| 24.2 | 5.1 |
| 24.9 | 12.24 |
| 25.2 | 8.76 |
| 26.1 | 6.04 |
| 26.4 | 9.41 |
| 26.8 | 12.98 |
| 27.3 | 5.63 |

In embodiments of the present disclosure, crystalline Form T11 of Deucravacitinib ESA is isolated.

Crystalline Form T11 of Deucravacitinib ESA may be hydrate form. The water content may of from about 2% to about 3%, for example 2.5% (w/w), as determined by Karl-Fischer.

Crystalline Form T11 of Deucravacitinib ESA may be characterized by each of the above characteristics alone or by all possible combinations, e.g., an XRPD pattern having peaks at 6.0, 7.0, 18.3, 19.5 and 21.0 degrees 2-theta±0.2 degrees 2-theta; an XRPD pattern as depicted in FIG. 14; and combinations thereof.

The disclosure encompasses crystalline Deucravacitinib:L-tartaric, i.e.

Deucravacitinib free base:L-tartaric acid complex. Crystalline Deucravacitinib:L-tartaric acid may be a co-crystal of Deucravacitinib and L-tartaric acid. Alternatively crystalline Deucravacitinib:L-tartaric acid may be a salt, i.e. Deucravacitinib:L-tartrate.

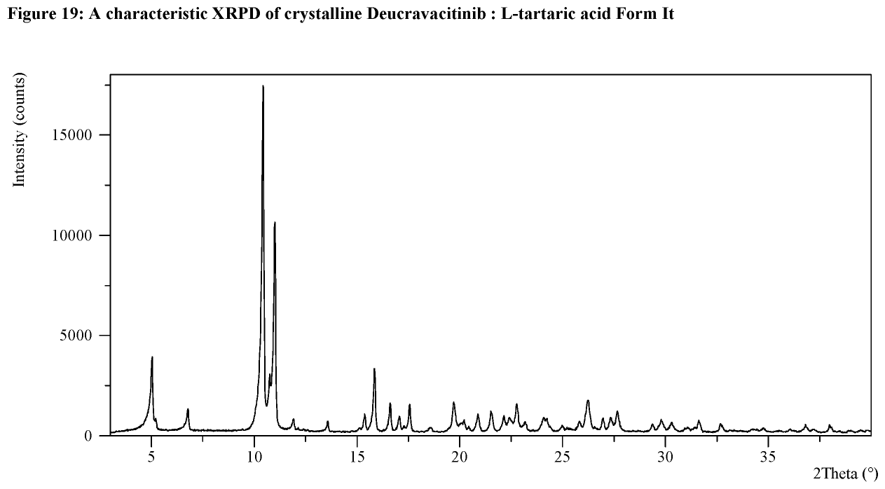

The disclosure further encompasses a crystalline form of Deucravacitinib:L-tartaric acid, designated form It. Crystalline Form It of Deucravacitinib:L-tartaric acid may be characterized by data selected from one or more of the following: an X-ray powder diffraction pattern substantially as depicted in FIG. 19; an X-ray powder diffraction patter having peaks at 10.4, 11.0, and 16.6 degrees 2-theta±0.2 degrees 2-theta; an X-ray powder diffraction pattern having peaks at 5.0, 10.4, 11.0, 16.6 and 19.7 degrees 2-theta±0.2 degrees 2-theta; and combinations of these data.

Crystalline Form It of Deucravacitinib:L-tartaric acid may be further characterized by an X-ray powder diffraction pattern having peaks at 5.0, 10.4, 11.0, 16.6 and 19.7 degrees 2-theta±0.2 degrees 2-theta, and also having any one, two, three or four additional peaks selected from 6.8, 17.6, 21.5 and 26.3 degrees 2-theta±0.2 degrees 2-theta.

Crystalline Form It of Deucravacitinib:L-tartaric acid may be characterized by an X-ray powder diffraction pattern having peaks as listed in Table 5. Crystalline Form It of Deucravacitinib:L-tartaric acid may be alternatively characterized by an XRPD pattern having the 2-theta values in Table 5 with an error value of ±0.2 degrees 2-theta, either with or without the respective relative intensity values):

TABLE 5

| Pos. [°2-theta] | Rel. Int. [%] |
|---|---|
| 5.0 | 22 |
| 5.2 | 3 |
| 6.8 | 6 |
| 10.4 | 100 |
| 10.8 | 16 |
| 11.0 | 60 |
| 11.9 | 4 |
| 13.6 | 3 |
| 15.4 | 5 |
| 15.9 | 18 |
| 16.6 | 8 |
| 17.1 | 5 |
| 17.6 | 8 |
| 19.7 | 8 |
| 20.2 | 3 |
| 20.4 | 2 |
| 20.9 | 5 |
| 21.5 | 6 |
| 21.6 | 5 |
| 22.1 | 5 |
| 22.4 | 4 |
| 22.8 | 8 |
| 23.2 | 3 |
| 24.1 | 4 |
| 24.2 | 4 |
| 25.0 | 2 |
| 25.8 | 3 |
| 26.3 | 9 |
| 26.9 | 4 |
| 27.3 | 4 |
| 27.6 | 6 |

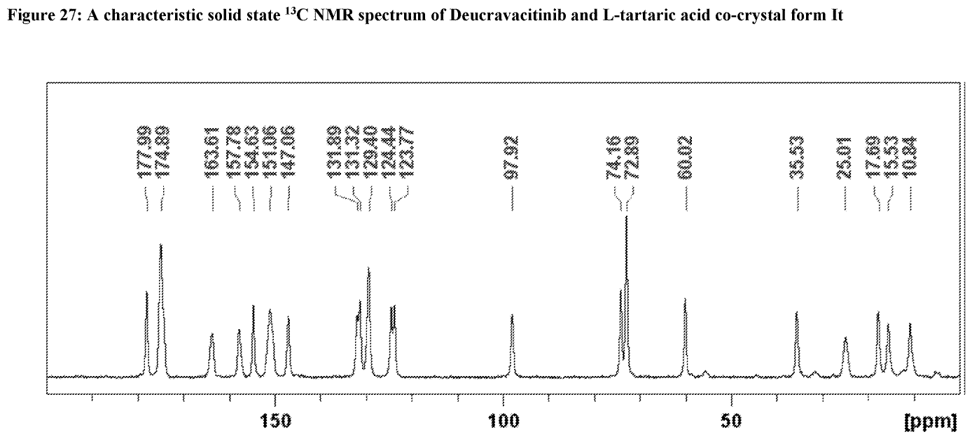

Deucravacitinib:L-tartaric acid Form It may alternatively or additionally be characterized by a solid state ¹³C spectrum having characteristic peaks at the range of 0-200 ppm at: 10.8, 15.5, 17.7, 25.0, 35.5, 60.0, 72.9, 74.2, 97.9, 123.8, 124.4, 129.4, 131.3, 131.9, 147.1, 151.1, 154.6, 157.8, 163.6, 174.9, and 178.0±2 ppm, or a solid-state ¹³C NMR spectrum substantially as depicted in FIG. 27.

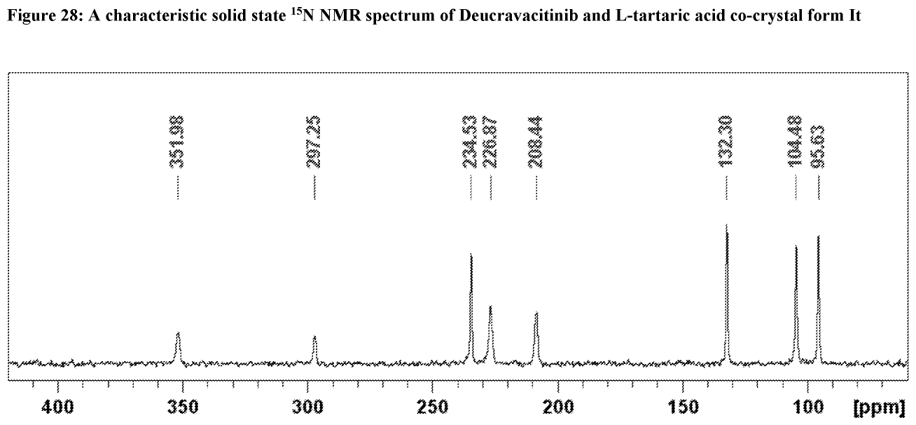

Deucravacitinib:L-tartaric acid Form It may be alternatively or additionally characterized by a solid state ¹⁵N NMR spectrum having characteristic peaks at the range of 0-400 ppm at: 95.6, 104.5, 132.3, 208.4, 226.9, 234.5, 297.3, and 352.0±2 ppm, or a solid-state ¹⁵N NMR spectrum substantially as depicted in FIG. 28.

In embodiments of the present disclosure, crystalline Form It of Deucravacitinib:L-tartaric acid is isolated.

Crystalline Form It of Deucravacitinib:L-tartaric acid may be an anhydrous form.

Crystalline Form It of Deucravacitinib:L-tartaric acid may be characterized by each of the above characteristics alone or by all possible combinations, e.g., an XRPD pattern having peaks at 5.0, 10.4, 11.0, 16.6 and 19.7 degrees 2-theta±0.2 degrees 2-theta; an XRPD pattern as depicted in FIG. 19; and combinations thereof.

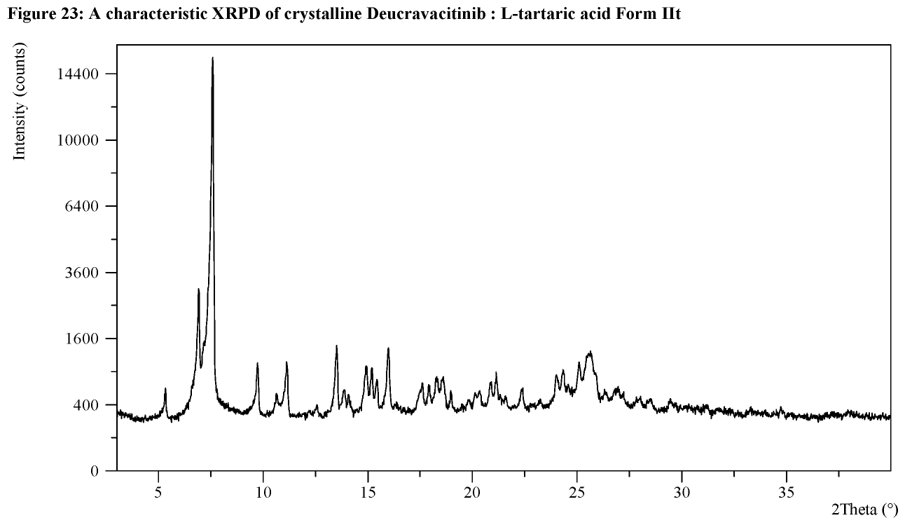

The disclosure further encompasses a crystalline form of Deucravacitinib:L-tartaric acid, designated form IIt. Crystalline Form IIt of Deucravacitinib:L-tartaric acid may be characterized by data selected from one or more of the following: an X-ray powder diffraction pattern substantially as depicted in FIG. 23; an X-ray powder diffraction pattern having peaks at 6.9, 7.6, 13.5, 15.2 and 17.9 degrees 2-theta±0.2 degrees 2-theta; and combinations of these data.

Crystalline Form IIt of Deucravacitinib:L-tartaric acid may be further characterized by an X-ray powder diffraction pattern having peaks at 6.9, 7.6, 13.5, 15.2 and 17.9 degrees 2-theta±0.2 degrees 2-theta, and also having any one, two, three or four additional peaks selected from 5.3, 9.7, 11.1 and 21.1 degrees 2-theta±0.2 degrees 2-theta.

In embodiments of the present disclosure, crystalline Form IIt of Deucravacitinib:L-tartaric acid is isolated.

Crystalline Form IIt of Deucravacitinib:L-tartaric acid may be a hydrate form.

Crystalline Form IIt of Deucravacitinib:L-tartaric acid may be characterized by each of the above characteristics alone or by all possible combinations, e.g., an XRPD pattern having peaks at 6.9, 7.6, 13.5, 15.2 and 17.9 degrees 2-theta±0.2 degrees 2-theta; an XRPD pattern as depicted in FIG. 23; and combinations thereof.

The disclosure encompasses crystalline Deucravacitinib:maleic acid, i.e.

Deucravacitinib free base:maleic acid is a distinct molecular species. Crystalline Deucravacitinib:maleic acid may be a co-crystal of Deucravacitinib and maleic acid. Alternatively crystalline Deucravacitinib:maleic acid may be a salt, i.e. Deucravacitinib maleate.

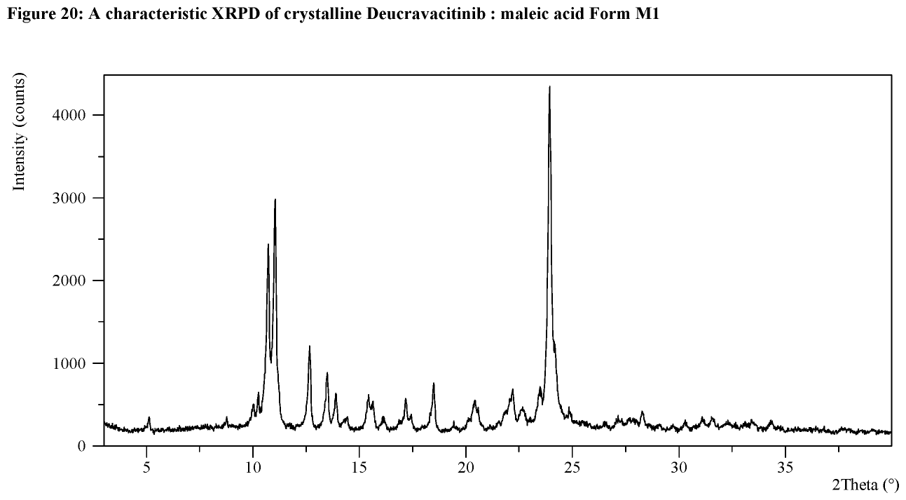

The disclosure further encompasses a crystalline form of Deucravacitinib:maleic acid, designated form M1. Crystalline Form M1 of Deucravacitinib:maleic acid may be characterized by data selected from one or more of the following: an X-ray powder diffraction pattern substantially as depicted in FIG. 20; an X-ray powder diffraction pattern having peaks at 10.7, 11.1, 18.5 and 23.9 degrees 2-theta±0.2 degrees 2-theta; and combinations of these data.

Crystalline Form M1 of Deucravacitinib:maleic acid may be further characterized by an X-ray powder diffraction pattern having peaks at 10.7, 11.1, 18.5 and 23.9 degrees 2-theta±0.2 degrees 2-theta, and also having any one, two, three or four additional peaks selected from 5.1, 12.7, 13.9 and 22.2 degrees 2-theta±0.2 degrees 2-theta.

In embodiments of the present disclosure, crystalline Form M1 of Deucravacitinib maleic acid is isolated.

Crystalline Form M1 of Deucravacitinib:maleic acid may be characterized by each of the above characteristics alone or by all possible combinations, e.g., an XRPD pattern having peaks at 10.7, 11.1, 18.5 and 23.9 degrees 2-theta±0.2 degrees 2-theta; an XRPD pattern as depicted in FIG. 20; and combinations thereof.

According to any aspect or embodiment of the present disclosure, any of the solid state forms of Deucravacitinib or Deucravacitinib hydrochloride described herein may be polymorphically pure or may be substantially free of any other solid state forms of Deucravacitinib or Deucravacitinib hydrochloride, respectively. In any aspect or embodiment of the present disclosure, any of the solid state forms of Deucravacitinib or Deucravacitinib hydrochloride may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other solid state forms of the subject compound (i.e. Deucravacitinib or Deucravacitinib hydrochloride respectively), preferably as measured by XRPD. Thus, any of the disclosed crystalline forms of Deucravacitinib described herein may be substantially free of any other solid state forms of Deucravacitinib, and may contain greater than about 80% (w/w), greater than about 90% (w/w), greater than about 95% (w/w), greater than about 98% (w/w), greater than about 99% (w/w), or about 100% of the subject solid state form of Deucravacitinib. Likewise, any of the disclosed crystalline forms of Deucravacitinib hydrochloride described herein may be substantially free of any other solid state forms of Deucravacitinib hydrochloride, and may contain greater than about 80% (w/w), greater than about 90% (w/w), greater than about 95% (w/w), greater than about 98% (w/w), greater than about 99% (w/w), or about 100% of the Deucravacitinib hydrochloride. As a further example, any of the disclosed crystalline forms of Deucravacitinib:L-tartaric acid described herein may be substantially free of any other solid state forms of Deucravacitinib:L-tartaric acid, and may contain greater than about 80% (w/w), greater than about 90% (w/w), greater than about 95% (w/w), greater than about 98% (w/w), greater than about 99% (w/w), or about 100% of the Deucravacitinib:L-tartaric acid; and may therefore contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other solid state forms Deucravacitinib:L-tartaric acid. The above solid state forms can be used to prepare other crystalline polymorphs of Deucravacitinib, Deucravacitinib salts or co-crystals and their solid state forms.

The present disclosure encompasses a process for preparing other solid state forms of Deucravacitinib, Deucravacitinib salts or co-crystals and their solid state forms thereof. The process includes preparing any one of the solid state forms of Deucravacitinib or Deucravacitinib HCl or Deucravacitinib salts by the processes of the present disclosure, and converting that form to a different form of Deucravacitinib, Deucravacitinib salt or co-crystal and solid state forms thereof.

The present disclosure provides the above described solid state forms, particularly crystalline polymorphs, of Deucravacitinib and of Deucravacitinib HCl and of Deucravacitinib salts for use in the preparation of pharmaceutical compositions comprising Deucravacitinib and/or crystalline polymorphs thereof. In embodiments, the above described solid state forms, particularly crystalline polymorphs, of Deucravacitinib and of Deucravacitinib HCl and crystalline polymorphs of Deucravacitinib salts are used to prepare oral dosage forms of Deucravacitinib.

The present disclosure also encompasses the use of the solid state forms, particularly crystalline polymorphs, of Deucravacitinib and of Deucravacitinib HCl and of Deucravacitinib salts of the present disclosure for the preparation of pharmaceutical compositions of crystalline polymorph Deucravacitinib or Deucravacitinib HCl and/or crystalline polymorphs thereof, particularly oral dosage forms.

The present disclosure includes processes for preparing the above mentioned pharmaceutical compositions. The processes include combining any one or a combination of the solid state forms, particularly crystalline polymorphs, of Deucravacitinib and of Deucravacitinib HCl or crystalline polymorphs of Deucravacitinib salts of the present disclosure with at least one pharmaceutically acceptable excipient.

Pharmaceutical combinations or formulations of the present disclosure contain any one or a combination of the solid state forms of Deucravacitinib or Deucravacitinib HCl or crystalline polymorphs of Deucravacitinib salts of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Deucravacitinib and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Deucravacitinib or Deucravacitinib HCl or of Deucravacitinib salts can be administered. Deucravacitinib may be formulated for administration to a mammal, in embodiments to a human, by injection or as ophthalmic solution for topical administration. Deucravacitinib can be formulated, for example, as a viscous liquid solution or suspension, such as a clear solution, for injection or as ophthalmic solution for topical administration. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The solid state forms, particularly crystalline polymorphs, of Deucravacitinib and of Deucravacitinib HCl or Deucravacitinib salts and the pharmaceutical compositions and/or formulations of Deucravacitinib, Deucravacitinib HCl or Deucravacitinib salts of the present disclosure can be used as medicaments, in embodiments for the treatment of patients with psoriasis.

The present disclosure also provides methods of treating of patients with psoriasis, by administering a therapeutically effective amount of any one or a combination of the solid state forms, particularly crystalline polymorphs, of Deucravacitinib and/or Deucravacitinib HCl or Deucravacitinib salts of the present disclosure, or at least one of the above pharmaceutical compositions and/or formulations, to a subject in need of the treatment.

Having described the disclosure with reference to certain or particular preferred embodiments, and/or illustrative examples other embodiments will become apparent to one skilled in the art from consideration of the specification. It will be apparent to those skilled in the art that many modifications, both to materials and methods, may be practiced without departing from the scope of the disclosure. The Examples are set forth to aid in understanding the disclosure but are not intended to, and should not be construed to limit its scope in any way.

### Powder X-Ray Diffraction (“XRPD”) Method

Sample was powdered in a mortar and pestle and applied directly on a silicon plate holder. The X-ray powder diffraction pattern was measured with Philips X′Pert PRO X-ray powder diffractometer, equipped with Cu irradiation source=1.54184 \acute{Å} (\acute{Å}ngström), X'Celerator (2.022° 2θ) detector. Scanning parameters: angle range: 3-40 deg., step size 0.0167, time per step 37 s, continuous scan.

The described peak positions were determined with or without using silicon powder as an internal standard in an admixture with the sample measured.

Solid State NMR (ssNMR) Method

Solid-state NMR spectra were acquired on Bruker Avance NEO 400 MHz NMR spectrometer equipped with 4.0 mm dual resonance HX CPMAS iProbe. Larmor frequencies of proton, carbon and nitrogen nuclei were 400, 100 and 40 MHz, respectively. ¹H and ¹³C NMR chemical shifts are reported relative to TMS (60.0 ppm). Chemical shifts were referenced using adamantane as an external reference for tetramethylsilane (TMS), setting the CH₂ signal to 38.48 ppm. ¹⁵N NMR chemical shifts are reported relative to liquid ammonia (δN 0.0 ppm). Samples were pressed in a 4.0 mm diameter ZrO₂ rotors and sealed with Kel-F caps. Spinning rates were 15 000 for ¹H NMR, 12 000 Hz for ¹³C NMR and 10 000 Hz for ¹⁵N NMR and ¹⁵N LG-CP-MAS experiments. For Lee-Goldberg cross-polarization experiments, ¹⁵N LG-CP MAS, a short excitation time of 200 μs was used to transfer the polarization to nitrogen atoms, in order to obtain selective detection of protonated nitrogen atoms.

### EXAMPLES

### Preparation of Starting Materials

Deucravacitinib can be prepared according to methods known from the literature, for example according to the disclosure in International Publications WO 2014074661, WO 2018183656 and/or WO 2021143498. Deucravacitinib HCl can be prepared by the process described in International Publications WO 2019232138 and/or WO 2021143430; or by any conventional method for salt preparation. Deucravacitinib form A can be prepared by the process described in International Publications WO 2018183656; or by any conventional method for salt preparation. Deucravacitinib HCl form CSII is described in WO 2021/129467, and can be obtained by the processes disclosed therein.

### Example A: Preparation of Starting Material Deucravacitinib HCl

Deucravacitinib base (5 grams) was suspended in 2-propanol (100 ml) at room temperature. Hydrochloric acid (36 wt %, 12 M, 1.3 ml) was added to the suspension and the suspension was heated to a temperature of about 50° C. The suspension was mixed for two days and then the solid was vacuum filtered. The obtained solid was vacuum dried at 80° C. to a constant mass, a crystalline solid obtained and was used in preparation of additional compounds as described herein below.

### Example 1: Preparation of Deucravacitinib Crystal Form B1

Deucravacitinib base (135 mg) was dissolved in acetic anhydride (3 ml) at a temperature of about 91° C. The obtained solution was left to cool and crystallization occurred at a temperature of about 63° C. The obtained suspension left to cool to room temperature and then maintained for additional 3 hours at room temperature while stirring. Then, the suspension was vacuum filtered and a sample was analyzed by XRPD; Deucravacitinib base form B1 was obtained. An XRPD pattern is shown in FIG. 1.

### Example 2: Preparation of Deucravacitinib Crystal Form B2

Deucravacitinib base (132 mg) was dissolved in a solvent mixture of acetic acid:water (ratio 2:1, total volume 3 ml) at room temperature. The obtained solution was slowly evaporated on a rotavapour at pressure of about 100 mbar for a period of about 1 hour, and a solid formed. A sample was analyzed by XRPD; Deucravacitinib base form B2 was obtained. An XRPD pattern is shown in FIG. 2.

### Example 3: Preparation of Deucravacitinib HCl Crystal Form H1

Deucravacitinib HCl (400 mg) was suspended in 4 ml of acetonitrile at room temperature. The Suspension was mixed for 2 days and then it was vacuum filtered. A Sample was analyzed by XRPD; Deucravacitinib HCl form H1 was obtained. An XRPD pattern is shown in FIG. 3.

### Example 4: Preparation of Deucravacitinib HCl Crystal Form H2

Deucravacitinib HCl form H1 (60 mg) was isothermally heated to a temperature of 173° C. at a heating rate of about 10° C./min and then cooled to temperature of about 30° C. The sample temperature (heating and cooling stages) was controlled using Anton Paar TCU100 Temperature Control Unit. During heating the XRPD was measured at certain points and form H2 was obtained from a temperature of about 75° C. to 173° C. After controlled cooling to temperature of about 30° C. a sample was analyzed by XRPD; Deucravacitinib HCl form H2 was obtained. An XRPD pattern is shown in FIG. 4.

### Example 5: Preparation of Deucravacitinib HCl Crystal Form H4

Deucravacitinib base (95.9 mg) and hydrochloric acid (0.110 ml) were suspended, and a few minutes after a solution was formed. Then, acetonitrile (2 ml) was added to the obtained solution. Crystallization was occurred after 1 day. The obtained solid was analyzed by XRPD. Deucravacitinib HCl form H4 was obtained. An XRPD pattern is shown in FIG. 5.

### Example 6: Preparation of Deucravacitinib HCl Crystal Form H8

Deucravacitinib HCl (80 mg) was dissolved in acetic acid (0.5 ml) at room temperature. 2-propanol (2 ml) was added dropwise and crystallization was occurred momentary. The obtained suspension was mixed for additional 2 hours and then the solid was vacuum filtered. The obtained solid was analyzed by XRPD. Deucravacitinib HCl form H8 was obtained. An XRPD pattern is shown in FIG. 6.

### Example 7: Preparation of Deucravacitinib HCl:L-Malic Acid Form H6

Deucravacitinib HCl form (756 mg, prepared according to Example A) and L-Malic acid (2 eq. 444 mg) were suspended in ethyl acetate (12 ml) at room temperature. The suspension was mixed for 9 days and the solid was vacuum filtered. The obtained solid was analyzed by XRPD. Deucravacitinib HCl and L-malic acid complex, form H6 was obtained. An XRPD pattern is shown in FIG. 7. Ratio between Deucravacitinib HCl and L-Malic acid was 1:1 (confirmed by HPLC).

### Example 8: Preparation of Deucravacitinib HCl:Urea Form H5

Deucravacitinib HCl (79 mg, prepared according to Example A) and urea (2 eq. 21 mg) were suspended in a solvent mixture of heptane with 10% of methanol (total: 1 ml) at room temperature. The obtained suspension was mixed for 4 days and the solid was vacuum filtered. The obtained solid was analyzed by XRPD, Deucravacitinib HCl and urea complex, form H5 was obtained. An XRPD pattern is shown in FIG. 8.

### Example 9: Preparation of Deucravacitinib HCl:Urea Form H5

Deucravacitinib HCl (79 mg, prepared according to Example A) and urea (2 eq. 21 mg) were suspended in 2-propanol (1 ml) at room temperature. The obtained suspension was mixed for 4 days and the solid was vacuum filtered. The obtained solid was analyzed by XRPD, Deucravacitinib HCl and urea complex, form H5 was obtained.

### Example 10: Preparation of Deucravacitinib HCl:Urea Form H5

Deucravacitinib HCl (79 mg, prepared according to Example A) and urea (2 eq. 21 mg) were suspended in ethyl acetate (1 ml) at room temperature. The obtained suspension was mixed for 4 days and vacuum filtered. The obtained solid was analyzed by XRPD, Deucravacitinib HCl and urea complex, form H5 was obtained.

### Example 11: Preparation of Deucravacitinib HCl:Urea Form H7

Deucravacitinib HCl (288 mg, prepared according to Example A) and urea (3 eq. 224 mg) were suspended in 2-propanol (4 ml) at room temperature. The obtained suspension was mixed for 8 days and the solid was vacuum filtered. The obtained solid was analyzed by XRPD, Deucravacitinib and urea complex, form H7 was obtained. An XRPD pattern is shown in FIG. 9.

### Example 12: Preparation of Deucravacitinib HCl and L-Malic Acid Complex Form H9

Deucravacitinib HCl and L-Malic acid complex (100 mg, form H6) was suspended in ethanol (96%, 1 ml) at room temperature. The suspension was mixed for 1 day and then the solid was vacuum filtered. The obtained solid was analyzed by XRPD and Deucravacitinib HCl and L-Malic acid complex form H9 was obtained. An XRPD pattern is shown in FIG. 10.

### Example 13: Preparation of Deucravacitinib HBr Form T1

Deucravacitinib (100 mg, free base form A) was suspended in 2-propanol (1 ml) at room temperature and HBr acid (1M, 0.28 ml) was added. The suspension was heated and a solution formed at temperature of about 51° C. The solution was additionally mixed for 15 minutes at temperature of about 51° C. and then it was allowed to cool to a temperature of about 0-5° C. The crystallization occurred at temperature of about 3° C. after about 4 hours of mixing, and the obtained suspension was vacuum filtered. The obtained solid was analyzed by XRPD and Deucravacitinib HBr form T1 was obtained. An XRPD pattern is shown in FIG. 11.

### Example 14: Preparation of Deucravacitinib HBr Form T1

Deucravacitinib (500 mg, free base form A) was suspended in acetone (5 ml) at room temperature and HBr acid (1 M, 1.4 ml) was added. The obtained suspension was heated and a solution formed at temperature of about 51° C. The solution was additionally mixed for 15 minutes at temperature of about 51° C. and allowed to cool to a temperature of about 0-5° C. The crystallization occurred at temperature of about 3° C. after about 4 hours of mixing and the obtained suspension was vacuum filtered. The obtained solid was analyzed by XRPD and Deucravacitinib HBr form T1 was obtained.

### Example 15: Preparation of Deucravacitinib HBr Form T2

Deucravacitinib HBr form T1 (2 mg) was placed in a pin hole aluminum pan. The sample was subjected to thermal treatment in DSC Discovery TA instruments, according to following steps:
1. Heating of the sample by heating rate 10° C./minute up to temperature of 187° C.,
2. Isothermal heating for 15 minutes at 187° C.,
3. Cooled to room temperature

The obtained solid was analyzed by XRPD. Deucravacitinib HBr form T2 was obtained. An XRPD pattern is shown in FIG. 12.

### Example 16: Preparation of Deucravacitinib EDSA Form T5

Deucravacitinib (500 mg, free base form A) 1,2 ethanedisulfonic acid (335.3 mg, dehydrate) were suspended in ethanol (96%, 5 ml) at room temperature. The obtained suspension was mixed for 1 day and the solid was vacuum filtered. The obtained solid was analyzed by XRPD and Deucravacitinib EDSA form T5 was obtained. An XRPD pattern is shown in FIG. 13.

### Example 17: Preparation of Deucravacitinib ESA Form T11

Deucravacitinib (300 mg, free base form A) was suspended in acetone (1.5 ml) at room temperature and ethanesulfonic acid (122.4 μl) was added. After about 10 minutes at room temperature, the suspension was dissolved and then it was allowed to cool to a temperature of about 0-5° C. After about 4 hours crystallization occurred and the obtained suspension was vacuum filtered. The obtained solid was analyzed by XRPD and Deucravacitinib ESA form T11 was obtained. An XRPD pattern is shown in FIG. 14.

### Example 18: Preparation of Deucravacitinib HCl:Urea Form H5

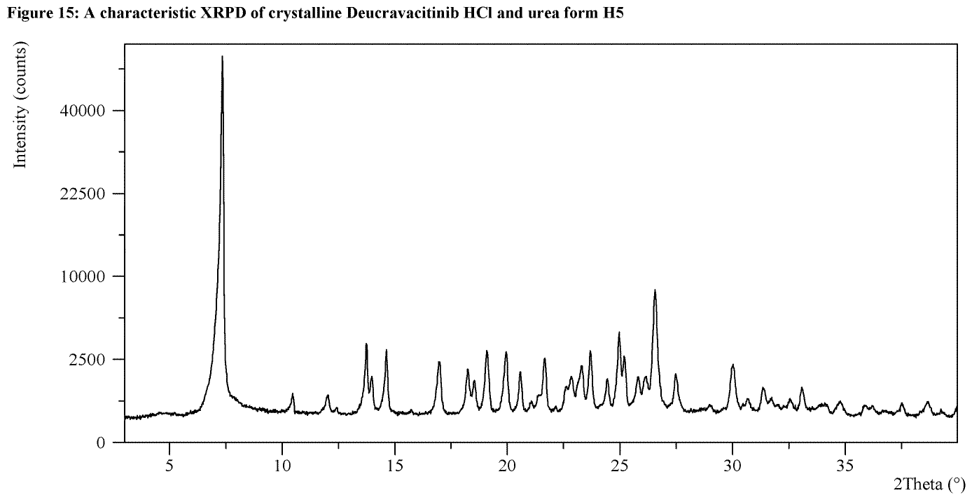

Deucravacitinib HCl (158 mg) and urea (62 mg) were dissolved in a solvent mixture of ethanol 96% and ethyl acetate and water (5% of water, 18.8% of ethanol 96% and 76.2% of ethyl acetate, total volume 5.3 ml) at temperature of about 60° C. the obtained solution was slowly allowed to cool to temperature of about 40° C. and ethyl acetate was added dropwise (21.2 ml). the crystallization occurred after addition of 5 ml ethyl acetate. The suspension was mixed at temperature of about 40° C. for 1 hour and then it was allowed to cool to room temperature. The suspension was additionally mixed for about 3 hours at room temperature and then the solid was vacuum filtered. The obtained solid was analyzed by XRPD and Deucravacitinib HCl-urea complex form H5 was obtained. An XRPD pattern is shown in FIG. 15.

### Example 19: Preparation of Deucravacitinib HCl Form H1

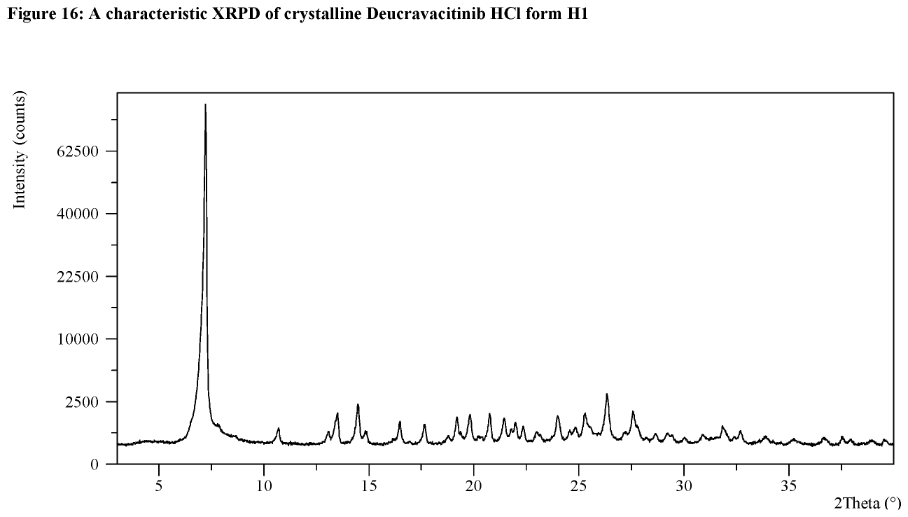

Deucravacitinib HCl form CSII (91 mg) was suspended in a solvent mixture of acetonitrile: water (5% of water, total volume 9 ml) at room temperature. HCl (12 M, 19.6 μL) was added and the obtained suspension was heated to a temperature of about 65° C. a solution formed at a temperature of about 64° C. and it was additionally mixed at that temperature for 15 minutes. After 15 minutes, the solution was allowed to cool to room temperature. Crystallization occurred at room temperature after 30 minutes and the suspension was additionally mixed for 3 hours. The suspension was vacuum filtered and dried at temperature of about 80° C. for 8 hours to a constant mass. The obtained solid was analyzed by XRPD and Deucravacitinib HCl form H1 (hydrate) was obtained. An XRPD pattern is shown in FIG. 16.

### Example 20: Preparation of Deucravacitinib and L-Malic Acid Complex Form Im

Deucravacitinib base (61 mg) and L-Malic acid (2 eq, 39 mg) were suspended in ethyl acetate (1 ml) at room temperature. The obtained suspension was mixed for 7 days and then the solid was vacuum filtered. The obtained solid was analyzed by XRPD and Deucravacitinib and L-Malic acid complex form Im was obtained. An XRPD pattern is shown in FIG. 18.

### Example 21: Preparation of Deucravacitinib and L-Tartaric Acid Complex Form It

Deucravacitinib base (59 mg) and L-Tartaric acid (2 eq, 41 mg) were suspended in of acetone (1 ml) at room temperature. The obtained suspension was mixed for 4 days and the solid was vacuum filtered. The obtained solid was analyzed by XRPD and Deucravacitinib and L-tartaric acid complex form It was obtained. An XRPD pattern is shown in FIG. 19.

### Example 22: Preparation of Deucravacitinib and Maleic Acid Complex Form M1

Deucravacitinib base (130 mg) and maleic acid (2 eq, 70 mg) were suspended in ethyl acetate (2 ml) at room temperature. The obtained suspension was mixed for 5 days and the solid was vacuum filtered. The obtained solid was analyzed by XRPD and Deucravacitinib base and maleic acid complex, form M1 was obtained. An XRPD pattern is shown in FIG. 20.

### Example 23: Preparation of Deucravacitinib and L-Malic Acid Complex Form IIm

Deucravacitinib base (613 mg) and malic acid (2 eq, 176.6 mg) were suspended in ethyl acetate (10 ml) at room temperature. The obtained suspension was mixed for 3 days and the solid was vacuum filtered. The obtained solid was analyzed by XRPD and Deucravacitinib base and malic acid complex, form IIm was obtained. An XRPD pattern is shown in FIG. 21.

### Example 24: Preparation of Deucravacitinib and L-Malic Acid Complex Form IIIm

Deucravacitinib and L-malic acid complex form Im (2 mg) was placed in a pin hole aluminum pan. The sample was subjected to thermal treatment in DSC Discovery TA instruments, according to following steps:
1. Heating of the sample by heating rate 10° C./minute up to temperature of 130° C.,
2. Isothermal heating for 15 minutes at 130° C.,
3. Cooled to room temperature by cooling rate 10° C./minute

The obtained solid was analyzed by XRPD. Deucravacitinib and L-malic acid complex form IIIm was obtained. An XRPD pattern is shown in FIG. 22.

### Example 25: Preparation of Deucravacitinib and L-Tartaric Acid Complex Form IIt

Deucravacitinib and L-tartaric acid complex form It (50 mg) was exposed to controlled relative humidity of 100% for 7 days. Then, the sample was analyzed by XRPD. Deucravacitinib and L-tartaric acid complex, form IIt was obtained. An XRPD pattern is shown in FIG. 23.

### Example 26: Preparation of Deucravacitinib and L-Tartaric Acid Complex Form IIt

Deucravacitinib and L-tartaric acid complex form It (50 mg) was placed in a sauna and exposed to water atmosphere for 1 day. Then, the sample was analyzed by XRPD. Deucravacitinib and L-tartaric acid complex, form IIt was obtained.

### Example 27: Stability Experiments

### Storage Stability at Different Relative Humidities

Samples of Deucravacitinib:L-tartaric acid Form It were subjected to conditions of different relative humidity (20%, 40%, 60% and 80%) at ambient temperature (25° C.). XRPD analysis was performed on the samples after 7 days and after 30 days. The results are shown in Table 6 below:

TABLE 6

| | Relative humidity | | | |
|---|---|---|---|---|
| XRPD analysis results | 20% | 40% | 60% | 80% |
| Deucravacitinib:L-tartaric acid | No | No | No | No |
| Form It (7 days) | change | change | change | change |
| Deucravacitinib L-tartaric acid | No | No | No | No |
| Form It (30 days) | change | change | change | change |

These results demonstrate that Deucravacitinib L-tartaric acid Form It is stable after exposure to high and low relative humidity conditions for at least 30 days.

### Grinding Experiments

Samples of Deucravacitinib L-tartaric acid Form It were subjected to strong grinding, and to solvent drop grinding in water, ethanol, ethyl acetate, 2-propanol, and acetone. Grinding was carried out on the sample alone, or in the presence of the solvents. In these experiments, about 20 mg of the sample is placed in a mortar and ground with a pestle for 3 minutes. The solvent, when used, was added to the crystalline material before grinding, in a volume of 10 microlitres. XRPD analysis performed on the sample after the grinding experiment, confirmed no change in the starting material (Table 7):

TABLE 7
Deucravacitinib L-tartaric acid Form It

| Condition | XRPD analysis results |
|---|---|
| Strong grinding | No change |
| Solvent-drop grinding | No change |
| (water) | |
| Solvent-drop grinding | No change |
| (ethanol) | |
| Solvent-drop grinding | No change |
| (ethylacetate) | |
| Solvent-drop grinding | No change |
| (2-propanol) | |
| Solvent-drop grinding | No change |
| (acetone) | |

The results demonstrate that Deucravacitinib L-tartaric acid Form It is resistant to polymorphic changes during grinding and are highly suitable for preparing pharmaceutical formulations.

### Exposure to Solvent Vapour

Samples of Deucravacitinib L-tartaric acid Form It were exposed to solvent vapour at 25° C. The solvents used were acetone, water, ethyl acetate, 2-propanol, and ethanol. After 7 days, the samples were measured by XRPD (Table 8):

TABLE 38
Deucravacitinib L-tartaric acid Form It

| Solvent Vapour | XRPD analysis results |
|---|---|
| Acetone | No change |
| Water | No change |
| Ethyl acetate | No change |
| 2-Propanol | No change |
| Ethanol | No change |

### Slurrying Experiments

Samples of Deucravacitinib L-tartaric acid Form It were slurried (stirred) each in acetone, and water. The samples were analyzed by XRPD after 1 day, and after 7 days (Table 9):

TABLE 9
Deucravacitinib L-tartaric acid Form It

| Solvent Vapour | XRPD analysis results |
|---|---|
| Acetone | No change |
| Ethyl acetate | No change |

### Thermal Stability

A sample of Deucravacitinib L-tartaric acid Form It was subjected to heating up to 100° C. for 30 minutes. XRPD analysis of the sample confirmed there to be no change in the starting material.

### Stability to Compression

Samples of Deucravacitinib:L-tartaric acid Form It were subjected to pressures of 19841 kg/m² and 39682 kg/m². XRPD analysis was performed on the samples after 1-5 minutes. The results are shown in Table 10 below:

TABLE 10

Pressure (kg/m²)

| XRPD analysis results | 19841 | 39682 |
|---|---|---|
| Deucravacitinib:L-tartaric acid | No change | No change |
| Form It (1-5 min) | | |

Accordingly Form It is stable under high pressure conditions, making it highly suitable for pharmaceutical processing.

### Example 28: Preparation of Ethyl 6-chloro-4-((2-methoxy-3-(1-methyl-1H-1,2,4-triazol-3-yl)phenyl)amino) pyridazine-3-carboxylate (Compound XIII)

2,2,6,6-tetramethylpiperidine (1.98 mL, 11.75 mmol) was added to a suspension of 2-methoxy-3-(1-methyl-1H-1,2,4-triazol-3-yl)aniline (2.0 grams, 9.79 mmol) and ethyl 4,6-dichloropyridazine-3-carboxylate (2.6 g, 11.75 mmol) in toluene (6 mL). The resultant suspension was heated to a temperature of about 110° C. and it was stirred at the same temperature until reaction completion. The resultant solution was cooled down to a temperature of about 20° C. and toluene (5 mL) was added, followed by water (9 mL) and suspension was formed. The obtained suspension was stirred 1 hour at a temperature of about 20° C. and the solid was then filtered. The filtered cake was washed with toluene (2×2 mL), then with water (2×2 mL) and was then the solid was dried under reduced pressure at a temperature of about 50° C. to afford intermediate (80% yield) as a crystalline solid.

### Example 29: Preparation of Ethyl 6-(cyclopropanecarboxamido)-4-((2-methoxy-3-(1-methyl-1H-1,2,4-triazol-3-yl)phenyl)amino)pyridazine-3-carboxylate (Compound XIV)

Into a reaction vessel were charged sequentially: ethyl 6-chloro-4-((2-methoxy-3-(1-methyl-1H-1,2,4-triazol-3-yl)phenyl)amino)pyridazine-3-carboxylate (Compound XIII, 0.50 grams, 1.29 mmol), cyclopropanecarboxamide (Compound II, 0.27 grams, 3.22 mmol) and potassium carbonate (0.82 grams, 5.93 mmol), followed by acetonitrile (2.3 mL) and toluene (3.8 mL). The resultant suspension was degassed and was added {(R)-1-[(Sp)-2-(Dicyclohexyl phosphino)ferrocenyl]ethyldi-tert-butylphosphine}[2-(2-amino-1,1-biphenyl)]palladium(II) methanesulfonate (commercial name: Josiphos SL-J009-1-G3-palladacycle, 0.024 grams, 0.026 mmol) followed by palladium acetate (0.009 grams, 0.039 mmol). The reaction slurry was heated to a temperature of about 75° C. and was stirred at this temperature until reaction completion. The reaction mixture was cooled down to a temperature of about 20° C. and was quenched by slow addition of 1:1 water/saturated ammonium chloride aq. solution (10.0 mL). Toluene (3.5 mL) was added and the reaction mixture was stirred at 20° C. for 0.5 hours. The suspension was filtered, and the filtered cake was washed with water (2×3.0 mL) and the solid was dried under reduced pressure at a temperature of about 80° C. to afford the titled intermediate compound (83% yield) as a crystalline solid.

The above described process can be done while utilizing (9,9-Dimethyl-9H-xanthene-4,5-diyl)bis(diphenylphosphane) (commercial name Xantphos, 0.017 grams, 0.029 mmol) instead of {(R)-1-[(Sp)-2-(Dicyclohexyl phosphino)ferrocenyl]ethyldi-tert-butylphosphine}[2-(2-amino-1,1-biphenyl)]palladium(II) methanesulfonate.

### Example 30: Preparation of 6-(cyclopropanecarboxamido)-4-((2-methoxy-3-(1-methyl-1H-1,2,4-triazol-3-yl)phenyl)amino)-N-(methyl-d3)pyridazine-3-carboxamide (Deucravacitinib)

A solution of Lithium bis(trimethylsilyl)amide, 1.3M in tetrahydrofuran (1.8 mL, 2.29 mmol, 2.0 molEq), was added slowly to a suspension of ethyl 6-(cyclopropanecarboxamido)-4-((2-methoxy-3-(1-methyl-1H-1,2,4-triazol-3-yl)phenyl)amino)pyridazine-3-carboxylate (0.5 grams, 1.14 mmol) and trideuteromethylamine hydrochloride (0.097 grams, 1.37 mmol) in dimethylformamide (2.5 mL). The resultant suspension was stirred at 20° C. until completion. Water (7.5 mL) was added slowly, followed by dropwise addition of hydrochloride (1.0 M, aqueous solution) until pH reached value 7.0. The suspension was then cooled down to a temperature of about 0° C. and was stirred at this temperature for about 1 hour. The suspension was filtered, and the obtained filter cake was washed with water (2×3 mL) and was dried under reduced pressure at to a temperature of about 80° C. to afford title compound (79% yield) as a crystalline solid.

### Example 31: Preparation of 6-chloro-4-((2-methoxy-3-(1-methyl-1H-1,2,3-triazol-3-yl)phenyl)amino)pyridazine-3-carboxamide (Compound XVII)

A solution of Lithium bis(trimethylsilyl)amide, 1.3M in tetrahydrofuran (1.4 mL, 1.82 mmol, 1.5 molEq), was added slowly to a suspension of ethyl 6-chloro-4-((2-methoxy-3-(1-methyl-1H-1,2,4-triazol-3-yl)phenyl)amino)pyridazine-3-carboxylate (Compound XIII, 0.5 grams, 1.21 mmol) and trideuteromethylamine hydrochloride (0.102 grams, 1.45 mmol) in tetrahydrofuran (5 mL). The resultant suspension was stirred at a temperature of about 20° C. until completion and was quenched by slow addition of 2:1 (v/v) water/saturated ammonium chloride aq. solution (7.5 mL). The layers were separated and the solvent from the upper layer was evaporated to dryness to afford the titled intermediate compound (75% yield) as a crystalline solid.

### Example 32: Preparation of 6-(cyclopropanecarboxamido)-4-((2-methoxy-3-(1-methyl-1H-1,2,4-triazol-3-yl)phenyl)amino)-N-(methyl-d3)pyridazine-3-carboxamide Deucravacitinib)

Into a reaction vessel were charged sequentially: 6-chloro-4-((2-methoxy-3-(1-methyl-1H-1,2,3-triazol-3-yl)phenyl)amino)pyridazine-3-carboxamide (Compound XVII), 0.50 grams, 1.29 mmol), cyclopropanecarboxamide (Compound II), 0.28 grams, 3.32 mmol) and potassium carbonate (0.85 grams, 6.11 mmol), followed by acetonitrile (2.3 mL) and toluene (3.8 mL). The resultant suspension was degassed and added {(R)-1-[(Sp)-2-(Dicyclohexyl phosphino)ferrocenyl]ethyldi-tert-butylphosphine}[2-(2-amino-1,1-biphenyl)]palladium(II) methanesulfonate was added followed by palladium acetate (0.009 grams, 0.027 mmol). The reaction slurry was heated to a temperature of about 75° C. and was stirred at this temperature until reaction completion. The reaction mixture was cooled down to a temperature of about 20° C. and was quenched by slow addition of 3:1 (v/v) water/saturated ammonium chloride aq. solution (7.0 mL). The suspension was filtered, and the filtered cake was washed with water (2×3.0 mL) and the solid was dried under reduced pressure at a temperature of about 80° C. to afford the titled compound (80% yield) as a crystalline solid.

Further aspects and embodiments of the present disclosure are set out in the numbered clauses below:

### Clauses—List A:

1. Crystalline Deucravacitinib HCl:L-Malic acid.
2. Crystalline Deucravacitinib HCl:L-Malic acid which is a co-crystal.
3. Crystalline Deucravacitinib HCl-L Malate.
4. A crystalline product according to Clause 1, 2, or 3, designated form H6, which is characterized by data selected from one or more of the following:
   a. an X-ray powder diffraction pattern having peaks at 6.7, 7.2, 8.5, 14.3 and 23.3 degrees 2-theta±0.2 degrees 2-theta;
   b. an XRPD pattern as depicted in FIG. 7; and
   c. combinations of these data.
5. A crystalline product according to any of Clauses 1, 2, 3 or 4, designated form H6, characterized by the XRPD pattern having peaks at 6.7, 7.2, 8.5, 14.3 and 23.3 degrees 2-theta±0.2 degrees 2-theta, and also having one, two, three or four additional peaks selected from 11.5, 13.4, 17.6, 17.9 and 27.7 degrees two theta±0.2 degrees two theta
6. A crystalline product according to any of Clauses 1, 2, 3, 4 or 5, designated form H6, wherein the crystalline form is an anhydrous form.
7. A crystalline product according to any of Clauses 1, 2, 3, 4, 5 or 6, designated form H6, which contains: no more than about 20%, no more than about 10%, no more than about 5%, no more than about 2%, no more than about 1% or about 0% of any other crystalline forms of Deucravacitinib HCl:L-Malic acid or crystalline HCl-L Malate.
8. A crystalline product according to any of Clauses 1, 2, 3, 4, 5 or 6, designated form H6, which contains: no more than about 20%, no more than about 10%, no more than about 5%, no more than about 2%, no more than about 1% or about 0% of amorphous Deucravacitinib HCl:L-Malic acid or crystalline HCl-L Malate.
9. A pharmaceutical composition comprising a crystalline product according to any of Clauses 1-8, and at least one pharmaceutically acceptable excipient.
10. Use of a crystalline product according to any of Clauses 1-8 for the preparation of a pharmaceutical composition and/or formulation, preferably wherein the pharmaceutical formulation is an oral formulation; for example a tablet, a capsule, etc.
11. A process for preparing the pharmaceutical composition according to Clause 9, comprising combining a crystalline product according to any of Clauses 1-8 with at least one pharmaceutically acceptable excipient.
12. A crystalline product according to any of Clauses 1-8, or a pharmaceutical composition according to Clause 9, for use as a medicament.
13. A crystalline product according to any of Claims 1-8, or a pharmaceutical composition according to Claim 9, for use in the treatment of psoriasis
14. A method of treating psoriasis comprising administering a therapeutically effective amount of a crystalline product according to any of Clauses 1-8, or a pharmaceutical composition according to Clause 9, to a subject in need of the treatment.
15. Use of a crystalline product according to any of Clauses 1-8, in the preparation of another solid state form of Deucravacitinib HCl:L-Malic or Deucravacitinib HCl-L-Malate.
16. A process for preparing a solid state form of Deucravacitinib HCl:L-Malic or Deucravacitinib HCl-L-Malate comprising preparing any one or a combination of a crystalline product according to any one of Clauses 1-8, and converting it to another a solid state form thereof.

Further aspects and embodiments of the present disclosure are set out in the numbered clauses below:

### Clauses—List B:

1. Crystalline Deucravacitinib HCl:Urea.
2. Crystalline Deucravacitinib HCl:Urea which is a co-crystal.
3. A crystalline product according to Clause 1 or 2, designated form H5, which is characterized by data selected from one or more of the following:
   a. an X-ray powder diffraction pattern having peaks at 7.3, 17.1, 20.1, 21.7 and 26.6 degrees 2-theta±0.2 degrees 2-theta;
   b. an XRPD pattern as depicted in FIG. 8; and
   c. combinations of these data.
4. A crystalline product according to any of Clauses 1, 2 or 3, designated form H5, characterized by the XRPD pattern having peaks at 7.3, 17.1, 20.1, 21.7 and 26.6 degrees 2-theta±0.2 degrees 2-theta, and also having one, two, three or four additional peaks selected from 14.7, 18.2, 20.6, 25.0 and 27.5 degrees two theta±0.2 degrees two theta 5. A crystalline product according to any of Clauses 1, 2, 3, or 4, designated form H5, wherein the crystalline form is an anhydrous form.
6. A crystalline product according to any of Clauses 1, 2, 3, 4 or 5, designated form H5, which contains: no more than about 20%, no more than about 10%, no more than about 5%, no more than about 2%, no more than about 1% or about 0% of any other crystalline forms of Deucravacitinib HCl:Urea.
7. A crystalline product according to any of Clauses 1, 2, 3, 4, 5 or 6, designated form H5, which contains: no more than about 20%, no more than about 10%, no more than about 5%, no more than about 2%, no more than about 1% or about 0% of amorphous Deucravacitinib HCl:Urea.
8. A pharmaceutical composition comprising a crystalline product according to any of Clauses 1-7, and at least one pharmaceutically acceptable excipient.
9. Use of a crystalline product according to any of Clauses 1-7 for the preparation of a pharmaceutical composition and/or formulation, preferably wherein the pharmaceutical formulation is an oral formulation; for example a tablet, a capsule, etc.
10. A process for preparing the pharmaceutical composition according to Clause 8, comprising combining a crystalline product according to any of Clauses 1-7 with at least one pharmaceutically acceptable excipient.
11. A crystalline product according to any of Clauses 1-7, or a pharmaceutical composition according to Clause 8, for use as a medicament.
12. A crystalline product according to any of Claims 1-7, or a pharmaceutical composition according to Claim 8, for use in the treatment of psoriasis
13. A method of treating psoriasis comprising administering a therapeutically effective amount of a crystalline product according to any of Clauses 1-7, or a pharmaceutical composition according to Clause 8, to a subject in need of the treatment.
14. Use of a crystalline product according to any of Clauses 1-7, in the preparation of another solid state form of Deucravacitinib HCl:Urea.
15. A process for preparing a solid state form of Deucravacitinib HCl:Urea comprising preparing any one or a combination of a crystalline product according to any one of Clauses 1-7, and converting it to another a solid state form thereof.

Further aspects and embodiments of the present disclosure are set out in the numbered clauses below:

### Clauses—List C:

1. Crystalline Deucravacitinib HCl:Urea.
2. Crystalline Deucravacitinib HCl:Urea which is a co-crystal.
3. A crystalline product according to Clause 1 or 2, designated form H7, which is characterized by data selected from one or more of the following:
   a. an X-ray powder diffraction pattern having peaks at 5.5, 8.4, 10.9, 11.4 and 14.5 degrees 2-theta±0.2 degrees 2-theta;
   b. an XRPD pattern as depicted in FIG. 9; and
   c. combinations of these data.
4. A crystalline product according to any of Clauses 1, 2 or 3, designated form H7, characterized by the XRPD pattern having peaks at 5.5, 8.4, 10.9, 11.4 and 14.5 degrees 2-theta±0.2 degrees 2-theta, and also having one, two, three or four additional peaks selected from 6.3, 13.8, 15.8, 17.6 and 22.3 degrees two theta±0.2 degrees two theta
5. A crystalline product according to any of Clauses 1, 2, 3, or 4, designated form H7, wherein the crystalline form is an anhydrous form.
6. A crystalline product according to any of Clauses 1, 2, 3, 4 or 5, designated form H7, which contains: no more than about 20%, no more than about 10%, no more than about 5%, no more than about 2%, no more than about 1% or about 0% of any other crystalline forms of Deucravacitinib HCl:Urea.
7. A crystalline product according to any of Clauses 1, 2, 3, 4, 5 or 6, designated form H7, which contains: no more than about 20%, no more than about 10%, no more than about 5%, no more than about 2%, no more than about 1% or about 0% of amorphous Deucravacitinib HCl:Urea.
8. A pharmaceutical composition comprising a crystalline product according to any of Clauses 1-7, and at least one pharmaceutically acceptable excipient.
9. Use of a crystalline product according to any of Clauses 1-7 for the preparation of a pharmaceutical composition and/or formulation, preferably wherein the pharmaceutical formulation is an oral formulation; for example a tablet, a capsule, etc.
10. A process for preparing the pharmaceutical composition according to Clause 8, comprising combining a crystalline product according to any of Clauses 1-7 with at least one pharmaceutically acceptable excipient.
11. A crystalline product according to any of Clauses 1-7, or a pharmaceutical composition according to Clause 8, for use as a medicament.
12. A crystalline product according to any of Claims 1-7, or a pharmaceutical composition according to Claim 8, for use in the treatment of psoriasis
13. A method of treating psoriasis comprising administering a therapeutically effective amount of a crystalline product according to any of Clauses 1-7, or a pharmaceutical composition according to Clause 8, to a subject in need of the treatment.
14. Use of a crystalline product according to any of Clauses 1-7, in the preparation of another solid state form of Deucravacitinib HCl:Urea.
15. A process for preparing a solid state form of Deucravacitinib HCl:Urea comprising preparing any one or a combination of a crystalline product according to any one of Clauses 1-7, and converting it to another a solid state form thereof.

Further aspects and embodiments of the present disclosure are set out in the numbered clauses below:

### Clauses—List D:

1. Crystalline Deucravacitinib HCl:L-Malic acid.
2. Crystalline Deucravacitinib HCl:L-Malic acid which is a co-crystal.
3. Crystalline Deucravacitinib HCl-L Malate.
4. A crystalline product according to Clause 1, 2, or 3, designated form H9, which is characterized by data selected from one or more of the following:
   a. an X-ray powder diffraction pattern having peaks at 7.9, 16.8, 19.9 and 25.3 degrees 2-theta±0.2 degrees 2-theta;
   b. an XRPD pattern as depicted in FIG. 10; and
   c. combinations of these data.
5. A crystalline product according to any of Clauses 1, 2, 3 or 4, designated form H9, characterized by the XRPD pattern having peaks at 7.9, 16.8, 19.9 and 25.3 degrees 2-theta±0.2 degrees 2-theta, and also having one, two, three or four additional peaks selected from 12.5, 13.0, 13.8 and 14.6 degrees two theta±0.2 degrees two theta
6. A crystalline product according to any of Clauses 1, 2, 3, 4 or 5, designated form H9, wherein the crystalline form is an anhydrous form.
7. A crystalline product according to any of Clauses 1, 2, 3, 4, 5 or 6, designated form H9, which contains: no more than about 20%, no more than about 10%, no more than about 5%, no more than about 2%, no more than about 1% or about 0% of any other crystalline forms of Deucravacitinib HCl:L-Malic acid or crystalline HCl-L Malate.
8. A crystalline product according to any of Clauses 1, 2, 3, 4, 5 or 6, designated form H9, which contains: no more than about 20%, no more than about 10%, no more than about 5%, no more than about 2%, no more than about 1% or about 0% of amorphous Deucravacitinib HCl:L-Malic acid or crystalline HCl-L Malate.
9. A pharmaceutical composition comprising a crystalline product according to any of Clauses 1-8, and at least one pharmaceutically acceptable excipient.
10. Use of a crystalline product according to any of Clauses 1-8 for the preparation of a pharmaceutical composition and/or formulation, preferably wherein the pharmaceutical formulation is an oral formulation; for example a tablet, a capsule, etc.
11. A process for preparing the pharmaceutical composition according to Clause 9, comprising combining a crystalline product according to any of Clauses 1-8 with at least one pharmaceutically acceptable excipient.
12. A crystalline product according to any of Clauses 1-8, or a pharmaceutical composition according to Clause 9, for use as a medicament.
13. A crystalline product according to any of Claims 1-8, or a pharmaceutical composition according to Claim 9, for use in the treatment of psoriasis
14. A method of treating psoriasis comprising administering a therapeutically effective amount of a crystalline product according to any of Clauses 1-8, or a pharmaceutical composition according to Clause 9, to a subject in need of the treatment.
15. Use of a crystalline product according to any of Clauses 1-8, in the preparation of another solid state form of Deucravacitinib HCl:L-Malic or Deucravacitinib HCl-L-Malate.
16. A process for preparing a solid state form of Deucravacitinib HCl:L-Malic or Deucravacitinib HCl-L-Malate comprising preparing any one or a combination of a crystalline product according to any one of Clauses 1-8, and converting it to another a solid state form thereof.

Further aspects and embodiments of the present disclosure are set out in the numbered clauses below:

### Clauses—List E:

1. Crystalline Deucravacitinib:L-Malic acid.
2. Crystalline Deucravacitinib:L-Malic acid which is a co-crystal.
3. Crystalline Deucravacitinib-L Malate.
4. A crystalline product according to Clause 1, 2, or 3, designated form Im, which is characterized by data selected from one or more of the following:
   d. an X-ray powder diffraction pattern having peaks at 5.0, 10.4, 11.0, 16.6 and 19.7 degrees 2-theta±0.2 degrees 2-theta;
   e. an XRPD pattern as depicted in FIG. 18; and
   f. combinations of these data.
5. A crystalline product according to any of Clauses 1, 2, 3 or 4, designated form Im, characterized by the XRPD pattern having peaks at 5.0, 10.4, 11.0, 16.6 and 19.7 degrees 2-theta±0.2 degrees 2-theta, and also having one, two, three or four additional peaks selected from 6.8, 17.6, 21.5 and 26.3 degrees two theta±0.2 degrees two theta
6. A crystalline product according to any of Clauses 1, 2, 3, 4 or 5, designated form Im, wherein the crystalline form is an anhydrous form.
7. A crystalline product according to any of Clauses 1, 2, 3, 4, 5 or 6, designated form Im, which contains: no more than about 20%, no more than about 10%, no more than about 5%, no more than about 2%, no more than about 1% or about 0% of any other crystalline forms of Deucravacitinib:L-malic acid or crystalline Deucravacitinib-L malate.
8. A crystalline product according to any of Clauses 1, 2, 3, 4, 5 or 6, designated form Im, which contains: no more than about 20%, no more than about 10%, no more than about 5%, no more than about 2%, no more than about 1% or about 0% of amorphous Deucravacitinib:L-malic acid or crystalline Deucravacitinib-L malate.
9. A pharmaceutical composition comprising a crystalline product according to any of Clauses 1-8, and at least one pharmaceutically acceptable excipient.
10. Use of a crystalline product according to any of Clauses 1-8 for the preparation of a pharmaceutical composition and/or formulation, preferably wherein the pharmaceutical formulation is an oral formulation; for example a tablet, a capsule, etc.
11. A process for preparing the pharmaceutical composition according to Clause 9, comprising combining a crystalline product according to any of Clauses 1-8 with at least one pharmaceutically acceptable excipient.
12. A crystalline product according to any of Clauses 1-8, or a pharmaceutical composition according to Clause 9, for use as a medicament.
13. A crystalline product according to any of Claims 1-8, or a pharmaceutical composition according to Claim 9, for use in the treatment of psoriasis
14. A method of treating psoriasis comprising administering a therapeutically effective amount of a crystalline product according to any of Clauses 1-8, or a pharmaceutical composition according to Clause 9, to a subject in need of the treatment.
15. Use of a crystalline product according to any of Clauses 1-8, in the preparation of another solid state form of Deucravacitinib:L-malic acid or crystalline Deucravacitinib-L malate.
16. A process for preparing a solid state form of Deucravacitinib:L-malic acid or crystalline Deucravacitinib-L malate comprising preparing any one or a combination of a crystalline product according to any one of Clauses 1-8, and converting it to another a solid state form thereof.

Further aspects and embodiments of the present disclosure are set out in the numbered clauses below:

### Clauses—List F:

1. Crystalline Deucravacitinib:L-tartaric acid.
2. Crystalline Deucravacitinib:L-tartaric acid, which is a co-crystal.
3. Crystalline Deucravacitinib-L tartrate.
4. A crystalline product according to Clause 1, 2, or 3, designated form It, which is characterized by data selected from one or more of the following:
   a. an X-ray powder diffraction pattern having peaks at 5.0, 10.4, 11.0, 16.6 and 19.7 degrees 2-theta±0.2 degrees 2-theta;
   b. an XRPD pattern as depicted in FIG. 19; and
   c. combinations of these data.
5. A crystalline product according to any of Clauses 1, 2, 3 or 4, designated form It, characterized by the XRPD pattern having peaks at 5.0, 10.4, 11.0, 16.6 and 19.7 degrees 2-theta±0.2 degrees 2-theta, and also having one, two, three or four additional peaks selected from 6.8, 17.6, 21.5 and 26.3 degrees two theta±0.2 degrees two theta.
6. A crystalline product according to any of Clauses 1, 2, 3, 4 or 5, designated form It, wherein the crystalline form is an anhydrous form.
7. A crystalline product according to any of Clauses 1, 2, 3, 4, 5 or 6, designated form It, which contains: no more than about 20%, no more than about 10%, no more than about 5%, no more than about 2%, no more than about 1% or about 0% of any other crystalline forms of Deucravacitinib:L-tartaric acid or crystalline-L tartrate.
8. A crystalline product according to any of Clauses 1, 2, 3, 4, 5 or 6, designated form It, which contains: no more than about 20%, no more than about 10%, no more than about 5%, no more than about 2%, no more than about 1% or about 0% of amorphous Deucravacitinib:L-tartaric acid or crystalline-L tartrate.
9. A pharmaceutical composition comprising a crystalline product according to any of Clauses 1-8, and at least one pharmaceutically acceptable excipient.
10. Use of a crystalline product according to any of Clauses 1-8 for the preparation of a pharmaceutical composition and/or formulation, preferably wherein the pharmaceutical formulation is an oral formulation; for example a tablet, a capsule, etc.
11. A process for preparing the pharmaceutical composition according to Clause 9, comprising combining a crystalline product according to any of Clauses 1-8 with at least one pharmaceutically acceptable excipient.
12. A crystalline product according to any of Clauses 1-8, or a pharmaceutical composition according to Clause 9, for use as a medicament.
13. A crystalline product according to any of Claims 1-8, or a pharmaceutical composition according to Claim 9, for use in the treatment of psoriasis
14. A method of treating psoriasis comprising administering a therapeutically effective amount of a crystalline product according to any of Clauses 1-8, or a pharmaceutical composition according to Clause 9, to a subject in need of the treatment.
15. Use of a crystalline product according to any of Clauses 1-8, in the preparation of another solid state form of Deucravacitinib:L-tartaric acid or Deucravacitinib-L-tartrate.
16. A process for preparing a solid state form of Deucravacitinib:L-tartaric acid or Deucravacitinib-L-tartrate comprising preparing any one or a combination of a crystalline product according to any one of Clauses 1-8, and converting it to another a solid state form thereof.

Further aspects and embodiments of the present disclosure are set out in the numbered clauses below:

### Clauses—List G:

1. Crystalline Deucravacitinib:maleic acid.
2. Crystalline Deucravacitinib:maleic acid, which is a co-crystal.
3. Crystalline Deucravacitinib maleate.
4. A crystalline product according to Clause 1, 2, or 3, designated form M1, which is characterized by data selected from one or more of the following:
   d. an X-ray powder diffraction pattern having peaks at 10.7, 11.1, 18.5 and 23.9 degrees 2-theta±0.2 degrees 2-theta;
   e. an XRPD pattern as depicted in FIG. 20; and
   f. combinations of these data.
5. A crystalline product according to any of Clauses 1, 2, 3 or 4, designated form M1, characterized by the XRPD pattern having peaks at 9.8, 10.7, 18.6 and 19.6 degrees 2-theta±0.2 degrees 2-theta, and also having one, two, three or four additional peaks selected from 5.1, 12.7, 13.9 and 22.2 degrees two theta±0.2 degrees two theta
6. A crystalline product according to any of Clauses 1, 2, 3, 4 or 5, designated form M1, which contains: no more than about 20%, no more than about 10%, no more than about 5%, no more than about 2%, no more than about 1% or about 0% of any other crystalline forms of Deucravacitinib:maleic acid or crystalline maleate.
7. A crystalline product according to any of Clauses 1, 2, 3, 4 or 5, designated form M1, which contains: no more than about 20%, no more than about 10%, no more than about 5%, no more than about 2%, no more than about 1% or about 0% of amorphous Deucravacitinib:Maleic acid or crystalline Maleate.
8. A pharmaceutical composition comprising a crystalline product according to any of Clauses 1-7, and at least one pharmaceutically acceptable excipient.
9. Use of a crystalline product according to any of Clauses 1-8 for the preparation of a pharmaceutical composition and/or formulation, preferably, wherein the pharmaceutical formulation is an oral formulation; for example a tablet, a capsule, etc.
10. A process for preparing the pharmaceutical composition according to Clause 8, comprising combining a crystalline product according to any of Clauses 1-7 with at least one pharmaceutically acceptable excipient.
11. A crystalline product according to any of Clauses 1-7, or a pharmaceutical composition according to Clause 8, for use as a medicament.
12. A crystalline product according to any of Claims 1-7, or a pharmaceutical composition according to Claim 8, for use in the treatment of psoriasis
13. A method of treating psoriasis comprising administering a therapeutically effective amount of a crystalline product according to any of Clauses 1-7, or a pharmaceutical composition according to Clause 8, to a subject in need of the treatment.
14. Use of a crystalline product according to any of Clauses 1-7, in the preparation of another solid state form of Deucravacitinib:maleic or Deucravacitinib maleate.
15. A process for preparing a solid state form of Deucravacitinib:maleic acid or Deucravacitinib-maleate comprising preparing any one or a combination of a crystalline product according to any one of Clauses 1-7, and converting it to another a solid state form thereof.

Further aspects and embodiments of the present disclosure are set out in the numbered clauses below:

### Clauses—List H:

1. Crystalline Deucravacitinib:L-Malic acid.
2. Crystalline Deucravacitinib:L-Malic acid which is a co-crystal.
3. Crystalline Deucravacitinib-L Malate.
4. A crystalline product according to Clause 1, 2, or 3, designated form IIm, which is characterized by data selected from one or more of the following:
   g. an X-ray powder diffraction pattern having peaks at 5.1, 9.5, 15.3 and 16.8 degrees 2-theta±0.2 degrees 2-theta;
   h. an XRPD pattern as depicted in FIG. 21; and
   i. combinations of these data.
5. A crystalline product according to any of Clauses 1, 2, 3 or 4, designated form IIm, characterized by the XRPD pattern having peaks at 5.1, 9.5, 15.3 and 16.8 degrees 2-theta±0.2 degrees 2-theta, and also having one, two, three or four additional peaks selected from 8.4, 17.9, 21.9 and 25.8 degrees two theta±0.2 degrees two theta
6. A crystalline product according to any of Clauses 1, 2, 3, 4 or 5, designated form IIm, wherein the crystalline form is solvate-hydrate form.
7. A crystalline product according to any of Clauses 1, 2, 3, 4, 5 or 6, designated form IIm, which contains: no more than about 20%, no more than about 10%, no more than about 5%, no more than about 2%, no more than about 1% or about 0% of any other crystalline forms of Deucravacitinib:L-malic acid or crystalline Deucravacitinib-L malate.
8. A crystalline product according to any of Clauses 1, 2, 3, 4, 5 or 6, designated form IIm, which contains: no more than about 20%, no more than about 10%, no more than about 5%, no more than about 2%, no more than about 1% or about 0% of amorphous Deucravacitinib:L-malic acid or crystalline Deucravacitinib-L malate.
9. A pharmaceutical composition comprising a crystalline product according to any of Clauses 1-8, and at least one pharmaceutically acceptable excipient.
10. Use of a crystalline product according to any of Clauses 1-8 for the preparation of a pharmaceutical composition and/or formulation, preferably wherein the pharmaceutical formulation is an oral formulation; for example a tablet, a capsule, etc.
11. A process for preparing the pharmaceutical composition according to Clause 9, comprising combining a crystalline product according to any of Clauses 1-8 with at least one pharmaceutically acceptable excipient.
12. A crystalline product according to any of Clauses 1-8, or a pharmaceutical composition according to Clause 9, for use as a medicament.
13. A crystalline product according to any of Claims 1-8, or a pharmaceutical composition according to Claim 9, for use in the treatment of psoriasis
14. A method of treating psoriasis comprising administering a therapeutically effective amount of a crystalline product according to any of Clauses 1-8, or a pharmaceutical composition according to Clause 9, to a subject in need of the treatment.
15. Use of a crystalline product according to any of Clauses 1-8, in the preparation of another solid state form of Deucravacitinib:L-malic acid or crystalline Deucravacitinib-L malate.
16. A process for preparing a solid state form of Deucravacitinib:L-malic acid or crystalline Deucravacitinib-L malate comprising preparing any one or a combination of a crystalline product according to any one of Clauses 1-8, and converting it to another a solid state form thereof.

Further aspects and embodiments of the present disclosure are set out in the numbered clauses below:

### Clauses—List I:

1. Crystalline Deucravacitinib:L-Malic acid.
2. Crystalline Deucravacitinib:L-Malic acid which is a co-crystal.
3. Crystalline Deucravacitinib-L Malate.
4. A crystalline product according to Clause 1, 2, or 3, designated form IIIm, which is characterized by data selected from one or more of the following:
   j. an X-ray powder diffraction pattern having peaks at 9.6, 10.5, 14.9, 24.6 and 26.1 degrees 2-theta±0.2 degrees 2-theta;
   k. an XRPD pattern as depicted in FIG. 22; and
   l. combinations of these data.
5. A crystalline product according to any of Clauses 1, 2, 3 or 4, designated form IIIm, characterized by the XRPD pattern having peaks at 9.6, 10.5, 14.9, 24.6 and 26.1 degrees 2-theta±0.2 degrees 2-theta, and also having one, two, three or four additional peaks selected from 5.2, 13.6, 17.9 and 18.5 degrees two theta±0.2 degrees two theta
6. A crystalline product according to any of Clauses 1, 2, 3, 4 or 5, designated form IIIm, wherein the crystalline form is an anhydrous form.
7. A crystalline product according to any of Clauses 1, 2, 3, 4, 5 or 6, designated form IIIm, which contains: no more than about 20%, no more than about 10%, no more than about 5%, no more than about 2%, no more than about 1% or about 0% of any other crystalline forms of Deucravacitinib:L-malic acid or crystalline Deucravacitinib-L malate.
8. A crystalline product according to any of Clauses 1, 2, 3, 4, 5 or 6, designated form IIIm, which contains: no more than about 20%, no more than about 10%, no more than about 5%, no more than about 2%, no more than about 1% or about 0% of amorphous Deucravacitinib:L-malic acid or crystalline Deucravacitinib-L malate.
9. A pharmaceutical composition comprising a crystalline product according to any of Clauses 1-8, and at least one pharmaceutically acceptable excipient.
10. Use of a crystalline product according to any of Clauses 1-8 for the preparation of a pharmaceutical composition and/or formulation, preferably wherein the pharmaceutical formulation is an oral formulation; for example a tablet, a capsule, etc.
11. A process for preparing the pharmaceutical composition according to Clause 9, comprising combining a crystalline product according to any of Clauses 1-8 with at least one pharmaceutically acceptable excipient.
12. A crystalline product according to any of Clauses 1-8, or a pharmaceutical composition according to Clause 9, for use as a medicament.
13. A crystalline product according to any of Claims 1-8, or a pharmaceutical composition according to Claim 9, for use in the treatment of psoriasis
14. A method of treating psoriasis comprising administering a therapeutically effective amount of a crystalline product according to any of Clauses 1-8, or a pharmaceutical composition according to Clause 9, to a subject in need of the treatment.
15. Use of a crystalline product according to any of Clauses 1-8, in the preparation of another solid state form of Deucravacitinib:L-malic acid or crystalline Deucravacitinib-L malate.
16. A process for preparing a solid state form of Deucravacitinib:L-malic acid or crystalline Deucravacitinib-L malate comprising preparing any one or a combination of a crystalline product according to any one of Clauses 1-8, and converting it to another a solid state form thereof.

Further aspects and embodiments of the present disclosure are set out in the numbered clauses below:

### Clauses—List J:

1. Crystalline Deucravacitinib:L-tartaric acid.
2. Crystalline Deucravacitinib:L-tartaric acid, which is a co-crystal.
3. Crystalline Deucravacitinib-L tartrate.
4. A crystalline product according to Clause 1, 2, or 3, designated form IIt, which is characterized by data selected from one or more of the following:
   m. an X-ray powder diffraction pattern having peaks at 6.9, 7.6, 13.5, 15.2 and 17.9 degrees 2-theta±0.2 degrees 2-theta;
   n. an XRPD pattern as depicted in FIG. 23; and
   o. combinations of these data.
5. A crystalline product according to any of Clauses 1, 2, 3 or 4, designated form IIt, characterized by the XRPD pattern having peaks at 6.9, 7.6, 13.5, 15.2 and 17.9 degrees 2-theta±0.2 degrees 2-theta, and also having one, two, three or four additional peaks selected from 5.3, 9.7, 11.1 and 21.1 degrees two theta±0.2 degrees two theta
6. A crystalline product according to any of Clauses 1, 2, 3, 4 or 5, designated form IIt, wherein the crystalline form is a hydrate form.
7. A crystalline product according to any of Clauses 1, 2, 3, 4, 5 or 6, designated form IIt, which contains: no more than about 20%, no more than about 10%, no more than about 5%, no more than about 2%, no more than about 1% or about 0% of any other crystalline forms of Deucravacitinib:L-tartaric acid or crystalline-L tartrate.
8. A crystalline product according to any of Clauses 1, 2, 3, 4, 5 or 6, designated form IIt, which contains: no more than about 20%, no more than about 10%, no more than about 5%, no more than about 2%, no more than about 1% or about 0% of amorphous Deucravacitinib:L-tartaric acid or crystalline-L tartrate.
9. A pharmaceutical composition comprising a crystalline product according to any of Clauses 1-8, and at least one pharmaceutically acceptable excipient.
10. Use of a crystalline product according to any of Clauses 1-8 for the preparation of a pharmaceutical composition and/or formulation, preferably wherein the pharmaceutical formulation is an oral formulation; for example a tablet, a capsule, etc.
11. A process for preparing the pharmaceutical composition according to Clause 9, comprising combining a crystalline product according to any of Clauses 1-8 with at least one pharmaceutically acceptable excipient.
12. A crystalline product according to any of Clauses 1-8, or a pharmaceutical composition according to Clause 9, for use as a medicament.
13. A crystalline product according to any of Claims 1-8, or a pharmaceutical composition according to Claim 9, for use in the treatment of psoriasis
14. A method of treating psoriasis comprising administering a therapeutically effective amount of a crystalline product according to any of Clauses 1-8, or a pharmaceutical composition according to Clause 9, to a subject in need of the treatment.
15. Use of a crystalline product according to any of Clauses 1-8, in the preparation of another solid state form of Deucravacitinib:L-tartaric acid or Deucravacitinib-L-tartrate.
16. A process for preparing a solid state form of Deucravacitinib:L-tartaric acid or Deucravacitinib-L-tartrate comprising preparing any one or a combination of a crystalline product according to any one of Clauses 1-8, and converting it to another a solid state form thereof.

1-62. (canceled)

63. Crystalline Deucravacitinib:L-Malic acid.

64. Crystalline Deucravacitinib:L-Malic acid according claim 63, which is a co-crystal.

65. Crystalline Deucravacitinib-L Malate.

66. A crystalline product according to claim 63, designated form Im, which is characterized by data selected from one or more of the following:
a. an X-ray powder diffraction pattern having peaks at 9.8, 10.7, 18.6 and 19.6 degrees 2-theta±0.2 degrees 2-theta;
b. an X-ray powder diffraction pattern having peaks at 9.8, 10.7, 18.6 and 19.6 degrees 2-theta±0.2 degrees 2-theta, and also having any one, two, three or four additional peaks selected from 4.4, 14.7, 24.0 and 24.5 degrees 2-theta±0.2 degrees 2-theta;
c. an XRPD pattern as depicted in FIG. 18;
d. combinations of these data.

67. A crystalline product according to claim 63, designated form Im, wherein the crystalline form is isolated.

68. A crystalline product according to claim 63, designated form Im, wherein the crystalline form is an anhydrous form.

69. A crystalline product according to claim 63, designated form Im, which contains no more than about 20% of any other crystalline forms of Deucravacitinib:L-Malic acid or crystalline Deucravacitinib-L malate.

70. A crystalline product according to claim 63, designated form Im, which contains no more than about 20% of amorphous Deucravacitinib:L-Malic acid or crystalline Deucravacitinib-L malate.

71. A pharmaceutical composition comprising a crystalline product according to claim 63 and at least one pharmaceutically acceptable excipient.

72. The pharmaceutical composition of claim 71, wherein the pharmaceutical composition is an oral formulation.

73. The pharmaceutical composition of claim 72, wherein the pharmaceutical composition is a tablet or a capsule.

74. A process for preparing a pharmaceutical composition comprising combining the crystalline product according to claim 63 with at least one pharmaceutically acceptable excipient.

75. A medicament comprising the crystalline product according to claim 63.

76. A method of treating psoriasis comprising administering a therapeutically effective amount of the crystalline product according to claim 63 to a subject in need of treatment.

77. A process for preparing a solid state form of Deucravacitinib:L-Malic acid or Deucravacitinib-L malate comprising preparing the crystalline product according to claim 63 and converting it to another solid state form thereof.
